(19) United States
(12) Patent Application Publication
(10) Pub. No.: US 2024/0194316 A1
(43) Pub. Date: Jun. 13, 2024

(54) FACILITATING ADHERENCE TO TASKS DESIGNED TO MAINTAIN OR IMPROVE HEALTH

(71) Applicant: Roche Diabetes Care, Inc., Indianapolis, IN (US)

(72) Inventor: Paul J. Galley, Cumberland, IN (US)

(73) Assignee: Roche Diabetes Care, Inc., Indianapolis, IN (US)

(21) Appl. No.: 18/443,926

(22) Filed: Feb. 16, 2024

Related U.S. Application Data
(63) Continuation of application No. PCT/US2022/040536, filed on Aug. 17, 2022.
(60) Provisional application No. 63/233,941, filed on Aug. 17, 2021.

(51) Int. Cl. G16H 20/00; G16H 10/60; G16H 80/00

(52) U.S. Cl. CPC G16H 20/00; G16H 10/60; G16H 80/00

(57) ABSTRACT

A user device (e.g., a mobile device) may determine a first notification visibility level for the user health-related event based on a context. The user device may receive data associated with the user health-related event. The user device may determine an adherence level for the user health-related event based on the data associated with the user health-related event. The user device may determine a second notification visibility level for the user health-related event based on comparison of the adherence level to a plurality of predefined adherence thresholds. The user device may communicate, using the second notification visibility level, a notification associated with the user health-related event via the user device upon detection of a triggering event. The user device may group, based on the context, one or more user health-related events into a notification group.

### CROSS-REFERENCE TO RELATED APPLICATIONS

This application claims priority to U.S. provisional patent application No. 63/233,941, filed Aug. 17, 2021 which is incorporated herein by reference in its entirety.

### TECHNICAL FIELD

The present invention relates generally to methods and systems for using predictive learning in diabetes disease progression analysis and treatment to increase user adherence.

### BACKGROUND

Devices are used to alert a user to perform health-related events. The user may be a person with diabetes (user), and the alert is a diabetes-related alert and/or notification. For example, the user's device may alert the user to perform a health-related event.

A person with diabetes may be required to track various different behaviors and related tasks. Adherence to these tasks enables the person with diabetes to understand how their health is impacted by certain behaviors and how to improve their health. The person with diabetes tracks the behaviors/tasks on a daily basis for optimal health management. Notifications (e.g., such as reminders) may be sent to the person with diabetes to remind them to perform the behaviors/tasks. Receiving too many notifications for the behaviors/tasks can result in a poor user experience for the person with diabetes and, in some cases, a regression or desensitization from the importance of the behaviors/tasks. For example, a person with diabetes who receives too many notifications could begin ignoring the notifications.

### SUMMARY

A computing device (e.g., a user device) is implemented to provide to display health-related (e.g., diabetes-related) events and/or tasks to a user. Notifications and/or alerts may be used to inform a user that a health-related event and/or task needs to be completed. A visibility of the notification and/or alert may be adjusted based on the user's adherence to the health-related event.

The user device (e.g., a health management application operating on the user device) may be configured to identify a user health-related event associated with a user. The user health-related event may be a diabetes related event. The user device may be configured to determine a context associated with the user health-related event. The context may include at least one of a time, a location, or a medical condition associated with the user health-related event. The user device may be configured to determine a first notification visibility level for the user health-related event. The first notification visibility level may be a measure of the visibility to the user of a notification associated with the user health-related event. The first notification visibility level may be determined based on the context. The user device may be configured to receive data associated with the user health-related event. At least a portion of the data is received by the user device upon scanning one or more of a bar code, a quick response (QR) code, a radio-frequency identification (RFID) tag, and/or a near field communication (NFC) tag.

The user device may be configured to determine an adherence level for the user health-related event based on the data associated with the user health-related event. The adherence level may include a range of adherence ratios for the user health-related event. The user health-related event may be associated with a plurality of adherence levels. Each of the plurality of adherence levels may be defined by an upper adherence threshold and a lower adherence threshold. The plurality of adherence levels may be adjustable based on one or more of a characteristic of the user, an age of the user health-related event, a characteristic of the user health-related event, a number of times adherence of the user health-related event has been logged within a predefined time period, and/or the frequency of the user health-related event.

The user device may be configured to compare the adherence level for the user health-related event to a plurality of predefined adherence thresholds. In response to comparing the adherence level to the plurality of predefined adherence thresholds, the user device may be configured to determine a second notification visibility level for the user health-related event. The second notification visibility level may be determined to be a higher visibility level than the first notification visibility level when the adherence level is below a first predefined adherence threshold associated with the first notification visibility level. The second notification visibility level may be determined to be a lower visibility level than the first notification visibility level when the adherence level is greater than a second predefined adherence threshold associated with the first notification visibility level. The user device may be configured to detect a triggering event associated with the user health-related event. The user device may be configured to communicate, using the second notification visibility level, the notification associated with the user health-related event via the user device.

The first and second notification visibility levels may include an intervention visibility level, a request input visibility level, a dashboard visibility level, and/or a hidden visibility level. The intervention visibility level may include communicating the notification to the mobile device of the user and to at least one other device. The request input visibility level may include requesting the user to log completion of the user health-related event. The dashboard visibility level may include displaying a list of the user health-related event and at least one other user health-related event to be completed in a given time period. The list of the user health-related event and at least one other user health-related event may be organized based on context. The hidden visibility level may include hiding the notification from the user. The request input visibility level may be selected as the first notification visibility level when the user health-related event is new to the user.

The user device may be configured to determine that the context associated with the user health-related event matches the context of at least one other user health-related event. The at least one other user health-related event may be a non-diabetes related event. The user device may be configured to group the user health-related event with the at least one other user health-related event in a notification group. The notification may indicate the user health-related event and the at least one other user health-related event.

A user device may be configured to log a first plurality of entries associated with a first user health-related event in a memory of the user device. The user device may be associated with a user. The first user health-related event may include one of a diabetes-related event or a non-diabetes related event. The user device may be configured to determine a first context associated with the first user health-related event. The first context may include at least one of a time or a location associated with the first user health-related event. The user device may be configured to determine that the plurality of entries indicate that the user is above an adherence threshold for the first user health-related event. The user device may be configured to log a second plurality of entries of a second user health-related event. The second user health-related event may include a diabetes-related event. The user device may be configured to determine a second context associated with the second user health-related event. The second context may include at least one of a time or a location associated with the second user health-related event. The user device may be configured to determine that the first context associated with the first user health-related event is within a predefined threshold of the second context associated with the second user health-related event. In response to determining that the context associated with the first user health-related event is similar to the context associated with the second user health-related event, the user device may be configured to group the first user health-related event with the second user health-related event in a notification group. The user device may be configured to detect a triggering event when the user is estimated to perform the first user-health related event. The user device may be configured to communicate, via an audible alert or a visible alert on the mobile device, an alert to the user to perform at least the second user health-related event.

The user device may be configured to adjust a visibility of the alert to perform the second user health-related event in response to a number of times the second user health-related event is logged within a predefined period of time and the adherence threshold. The alert may be communicated based on the triggering event being detected for the first user-health related event. The first user-health related event being above the adherence threshold may cause the visible alert or audible alert to fail to include information related specifically to the first user-health related event. The alert may be communicated at a notification visibility level which is determined based on the context. The notification visibility level comprises an intervention visibility level, a request input visibility level, a dashboard visibility level, or a hidden visibility level. The request input visibility level may be selected as the first notification visibility level when the user health-related event is new to the user.

### BRIEF DESCRIPTION OF THE DRAWINGS

FIG. 1 is a perspective view of a representative environment for monitoring or treating a diabetic condition.

FIG. 2 is a block diagram of an example computing device.

FIG. 3 is a block diagram of an example blood glucose monitoring device.

FIG. 4 is a block diagram of an example blood glucose measuring (BGM) device.

FIG. 5 is a block diagram illustrating an example of an insulin pump.

FIG. 6 shows a flowchart of an example process for determining a notification visibility level of a health-related event.

FIG. 7 shows a flowchart of another example process for determining a notification visibility level of a health-related event.

FIG. 8 shows a flowchart of an example process for adjusting a notification visibility level of a health-related event.

FIG. 9 shows a flowchart of an example process of grouping health-related events into a notification group.

FIG. 10 shows a flowchart of an example process for determining whether to send an alert to a health-related event notification group.

FIG. 11 shows a flowchart of another example process for adjusting a notification visibility level of a health-related event notification group.

### DETAILED DESCRIPTION

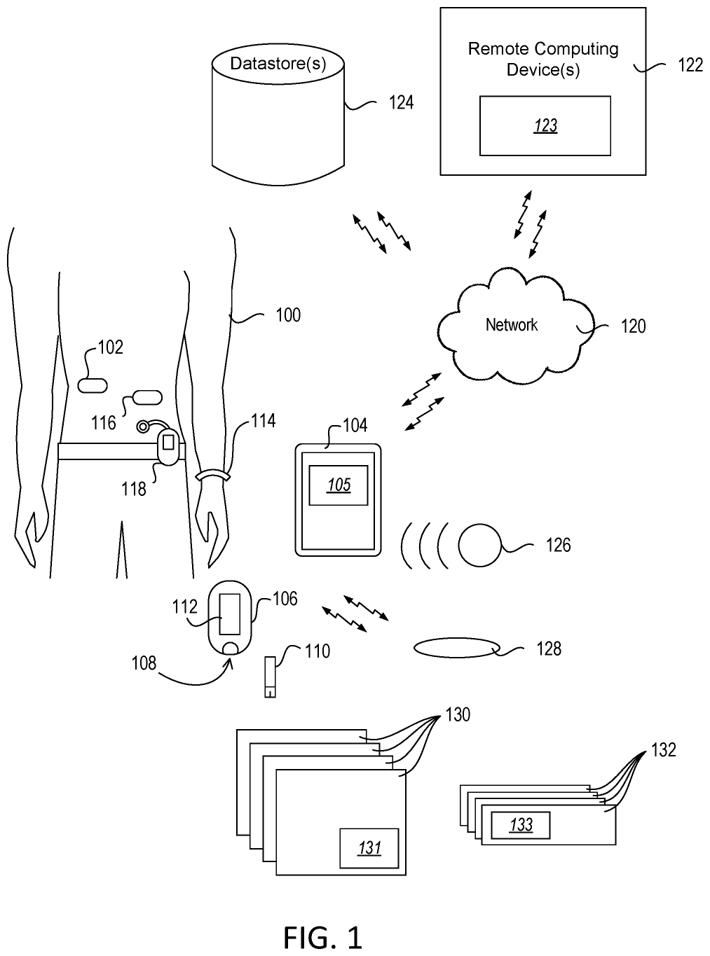

FIG. 1 is a perspective view of a representative environment for monitoring or treating a diabetic condition. As shown in FIG. 1, a user 100 with diabetes uses one or more blood glucose monitoring devices to help monitor or treat the diabetic condition. The diabetic condition includes a metabolic syndrome, pre-diabetes, type 1 diabetes, type 2 diabetes, or gestational diabetes. The user 100 may be in an extreme diabetic state, such as hypoglycemia or hyperglycemia, when the blood glucose level of the user 100 is above or below a threshold blood glucose level. In the embodiment of FIG. 1, the user 100 uses a blood glucose monitoring device to monitor blood glucose levels.

As used herein, the term “blood glucose monitoring device” refers to any device that detects and reports a level of glucose in the blood of the user, either through direct measurement of the blood or through an indirect detection process. A blood glucose level is also referred to as a blood sugar level. Examples of blood glucose monitoring devices include, but are not strictly limited to, continuous glucose monitoring devices, flash glucose monitoring devices, and blood glucose meters that provide a single measurement of blood glucose levels from a blood sample in a “spot” monitoring process. FIG. 1 depicts examples of blood glucose monitoring devices that are described in more detail below.

In some embodiments, the blood glucose monitoring device is a continuous glucose monitor (CGM) 102. The CGM 102 includes a subcutaneous sensor that is used to sense and monitor the amount of glucose in interstitial fluid of the user 100. The CGM 102 includes a transmitting device that is located directly over the sensor that wirelessly powers the data transfer from the sensor. The CGM 102 periodically communicates data indicating the blood glucose levels of the user 100 to an external device, such as a mobile device 104, for computing or storing the blood glucose levels of the user 100.

As used herein, the term “mobile device” refers to any mobile electronic device that is capable of moving with a user as the user changes locations. Example mobile devices include mobile phones, smartphones, wearable devices, tablets, laptops, notebook computers, personal digital assistants (PDAs), and any other mobile electronic device that is capable of moving with a user. Some embodiments of the mobile device incorporate the blood glucose monitor into an integrated device.

Some embodiments of the mobile device 104 operate as a CGM controller device. Though the mobile device 104 is provided as an example of a device with which the CGM 102 communicates, the CGM 102 may communicate with other dedicated CGM controller devices for providing similar functionality that is described herein for the mobile device 104. The CGM 102 processes the blood glucose data for providing alerts, or the blood glucose data is processed at the mobile device 104, or another CMG controller device, an alert indicator is communicated to the CGM 102.

In some embodiments, the blood glucose monitoring is performed by flash glucose monitoring (FGM). The FGM includes a subcutaneous sensor 103 that is used to sense and monitor the amount of glucose in interstitial fluid of the user 100. A separate reader device, such as the mobile device 102 or another reader device, receives the blood glucose information from the sensor when the device is within the RF range of the sensor 103. The sensor 103 transmits an instantaneous blood glucose level or a graphical trend of the blood glucose level to the reader device for display.

The user 100 uses a blood glucose meter (BGM) 106 as a blood glucose monitoring device to monitor blood glucose levels. The BGM 106 includes port 108 that receives a blood glucose measurement strip 110. The user 100 deposits a sample of blood on the blood glucose measurement strip 110. The BGM 106 analyzes the sample and measure the blood glucose level in the sample. The blood glucose level measured from the sample is displayed on a display 112 of the BGM 106 or communicated to an external device, such as the mobile device 104.

The blood glucose level measured by the BGM 106 or computed using data received from the CGM 102 is used to treat the diabetic condition of the user 100. For example, the user 100 uses an ambulatory non-durable insulin pump 116 or an ambulatory durable insulin pump 118 to treat the diabetic condition with insulin. The mobile device 104 determines an amount of insulin to be administered to the user 100 and the insulin pump 116, 118 receives instructions from the mobile device 104 to deliver a predetermined amount of insulin to the user 100. The insulin pump 116, 118 receives other information from the mobile device 104, such as mealtime information or exercise information of the user 100. The insulin pump 116, 118 determines the amount of insulin to administer based on the received information from the mobile device 104. The insulin pump 116, 118 communicates information to the mobile device 104. The information communicated to the mobile device 104 includes an amount of insulin delivered to the user 100, corresponding times of delivery, or a pump status (e.g., battery status, insulin status, or another status of a portion of the pump).

In some embodiments, the user 100 wears a fitness tracker 114. The fitness tracker 114 is a fitness band (e.g., as shown in FIG. 1), a smart watch, or another wearable device. The fitness tracker 114 measures and accounts for the user's activity or lack thereof. The fitness tracker 114 is used to measure activity, such as walking, motion, running, sleeping, being inactive, bicycling, exercising on an elliptical trainer, and the like. The data (e.g., activity data) collected by the fitness tracker 114 is transferred to and viewable on a computing device (e.g., such as the mobile device 104).

In some embodiments, the user 100 receives pre-packaged meals 130 to consume during the testing period. Each of the pre-packaged meals 130 includes a meal machine readable optical label 131. The meal machine readable optical label 131 is a bar code, a QR code, or some other unique identifier. The user 100 scans the meal machine readable optical label 131 prior to consuming the meal, for example, to confirm that the respective pre-packaged meal 130 was consumed.

In some embodiments, the user 100 receives pre-packaged medication doses 132 to take during the testing period. Each of the pre-packaged medication doses 132 includes a medication machine readable optical label 133. The medication machine readable optical label 133 is a bar code, a QR code, or some other unique identifier. The user 100 scans the medication machine readable optical label 133 prior to taking the medication dose, for example, to confirm that the respective pre-packaged medication dose 132 was taken.

In some embodiments, the user 100 uses a smart plate 128 to consume meals. The smart plate 128 includes one or more sensors configured to determine how much of the meal (e.g., the pre-packaged meal) that the user 100 has eaten. The smart plate 128 is configured to scan the meal machine readable optical label 131 of a pre-packaged meal 130. For example, the smart plate 128 includes a camera or barcode scanner.

The mobile device 104 communicates with the insulin pump 116, 118, the CGM 102, the BGM 106, the fitness tracker 114, and the smart plate 128 using wired or wireless communications. The mobile device 104, the CGM 102, a CGM controller, the BGM 106, the fitness tracker 114, the smart plate 128, and the insulin pump 116, 118 are collectively referred to as user devices. The mobile device 104 communicates with the insulin pump 116, 118, the CGM 102, the BGM 106, the fitness tracker 114, and the smart plate 128 using the same or different wireless protocols. For example, the mobile device 104 communicates with the insulin pump 116, 118, the CGM 102, the BGM 106, the fitness tracker 114, and the smart plate 128 using BLUETOOTH®, near field communication (NFC), THREAD®, WIFI®, ZIGBEE®, WI-MAX®, a cellular communication protocol, a proprietary wireless communication protocol, or another radio frequency (RF) communication protocol.

The mobile device 104 receives data and stores data for assisting in monitoring or treating the diabetic condition. The mobile device 104 receives input from the user 104 via a user interface being provided on a display. The mobile device 104 receives input via hard buttons or soft buttons provided on the display.

The mobile device 104 is configured to determine the device's location. For example, the mobile device 104 is able to determine the geolocation (e.g., latitude and longitude) of the device using signals from a global positioning system (GPS) or triangulation via cellular communications. The mobile device 104 determines a relative location using an RF beacon device 126. The RF beacon device 126 communicates a unique identifier via a short-range wireless communication, such as a BLUE TOOTH® low energy (BLE) beacon or an NFC beacon. The mobile device 104 receives the RF beacon and perform a lookup in a database (e.g., in information from the datastores 124) to determine a relative location associated with the unique identifier. For example, the mobile device 104 determines that the RF beacon indicates that the device is in a particular room in a home or building, on a certain floor in a building, close to a predefined object, or is within the RF range of a beacon associated with another object or location.

Some embodiments of the mobile device 104 include one or more sensors for detecting a relative position of the device or information about the user 100. The mobile device 104 detects a movement or a change in orientation. Based on the movement or change in orientation (or lack thereof) of the mobile device 104 over a period of time, the mobile device 104 detects that the user 100 is standing, sitting, or lying down. The mobile device 104 detects that the user 100 is exercising when the movement or a change in orientation is greater than a threshold for a period of time. The mobile device 104 detects the heartrate of the user 100 using a heartrate sensor. Based on the heartrate and the movement of the user 100 over a period of time, the mobile device 104 detects whether the user 100 is asleep or awake. The information about the mobile device 104 or the user 100 is used to provide information about or treat the diabetic condition.

The mobile device 104 provides information to the user 100 about the user's diabetic condition. For example, the mobile device 104 provides blood glucose levels, provides meal related information, provides exercise-related information, generates graphs and other graphical user interfaces for display, or generates alerts that are provided to the user 100. For example, the mobile device 104 measures the blood glucose level of the user 100 and provide an alert when the blood glucose level of the user 100 has reached a threshold for an extreme diabetic state (e.g., hypoglycemia or hyperglycemia). The alerts provided by the mobile device 104 are audible or non-audible alerts. The non-audible alerts are provided as a vibration, a flashing of the screen, or a flashing of an LED on the mobile device 104. The alerts also, or alternatively, are provided by an external device based on a communication from the mobile device 104. Some embodiments of the mobile device 104 include an electric motor for providing on-body vibration alerts or a speaker for providing audible alerts in response to data indications or triggers identified in the monitored blood glucose data.

The mobile device 104 communicates with other devices directly via a wired communication or a short-range wireless communication (e.g., WI-FI®, BLUETOOTH®, BLE, NFC, or another suitable short-range wireless communication). The mobile device 104 communicates indirectly with remote computing devices 122 or datastores 124 via a network 120 (e.g., using a WI-FI® network, a cellular network, a WI-MAX® network, or another wired or wireless network). The network 120 is a wired or wireless network. The network 120 is used to communicate over the Internet to other devices.

The mobile device 104 communicates with the remote computing devices to generate user interfaces for display on the mobile device 104, perform remote computation, or to otherwise control a remote computing device. For example, the mobile device 104 provides a user interface via an application or web browser that is generated at a remote computing device 122. The mobile device 104 generates instructions for providing alerts via remote computing devices 122 based on information received from the user 100, the CGM 102, the BGM 106, or the insulin pump 116, 118. Example remote computing devices 122 to which the mobile device 104 sends communications for performing alerts include a remote computer (e.g., a server, a laptop, or other computer), an external speaker, an external display device (e.g., television, monitor, or another device having an external display), a home automation system, remote telecommunications devices (e.g., for sending text or voice communications to an emergency contact over a telecommunications network), or another remote computing device.

The mobile device 104 communicates with the datastores 124 to store information or retrieve information. The information includes information related to the user 100, the CGM 102, the BGM 106, the fitness tracker 114, the smart plate 128, and/or the insulin pump 116, 118. For example, the mobile device 104 receives treatment information associated with the user 100 as input or receive blood glucose information from the CGM 102 or the BGM 106 and send the information to the datastores 124 via the network 120. Stored information is retrieved from the datastores 124 for treatment of the diabetic condition of the user. For example, the mobile device 104 retrieves an amount of insulin delivered to the user 100 or corresponding times of delivery. The datastores 124 include one or more remote storage locations, which are collectively referred to as cloud storage. For example, the datastores 124 store information regarding one or more personal characteristics of the user 100 (e.g., the user's age or gender) or one or more alert profiles for an alert.

One or more of the user devices (e.g., such as the mobile device 104) are configured to provide notifications (e.g., such as an alert) associated with a health-related event (e.g., such as a diabetes-related event) to a user. The notifications are used to inform a user that the health-related event needs to be completed. When the user completes the health-related event, the user may be required to log completion of the health-related event, for example, using the mobile device 104. The health-related event may include a task, a scheduled event, an unscheduled event, a habit, and/or the like. The health-related event may include consuming a meal, performing an activity (e.g., such as a type of exercise), taking a dose of medication, logging a medical reading (e.g., a blood glucose level, a body temperature, a heart rate, a blood pressure, an oxygen saturation level), logging sleep information, prescription status (e.g., prescription ordered, prescription filled, etc.), vaccination status, medical visits/checkups (e.g., dental visit, primary care visit, specialist visit, eye exam, etc.) and/or the like.

A user may use various methods to log completion of a health-related event. For example, a user may manually log completion, scan an identifier associated with the health-related event, and/or the like. The mobile device 104 may execute a mobile application 105 that enables manual recording health-related event completion. The mobile application 105 may be referred to herein as a health management application, a mobile health application, and/or the like.

Health-related events may be stored alongside calendar information, for example, to support marking health-related events as completed outside of a particular app, but accessible via API. Health-related events that are completed in advance of a specific reminder or as part of a daily to-do list may be excluded from reminder activation and marked as completed on a daily (or contextualized) to-do list. Each health-related event may have its own acceptance criteria where it can be counted as completed. The acceptance criteria may include a certain time window within which the data can be logged and/or a range of values or content that may be accepted as a completed health-related event.

Less frequent (e.g., ad-hoc, monthly, quarterly or annual) but recurring health-related events such as dental visits, medical checkups, eye exams, foot exams may be assessed for adherence purposes.

Notifications may also be used to inform the user of a problem with a device for monitoring or treating a diabetic condition. To better ensure that the user receives the notifications regarding the health-related events or the status of the user's devices for monitoring or treating the diabetic condition, an alert may be modified based on user input, lack of user input, user-specific characteristics, or the user environment.

The mobile device 104 (e.g., mobile application 105) may be configured to determine a notification visibility level of a health-related event. The notification visibility level of the health-related event may be a measure of the visibility to the user of a notification associated with the health-related event. An initial notification visibility level may be determined based on the context of the health-related event. The mobile device 104 may display the notification to the user according to the initial notification visibility level until enough adherence data is collected to update the initial notification visibility level. For example, mobile device 104 may be configured to receive data associated with the health-related event. The mobile device 104 may be configured to determine an adherence level for the user health-related event based on the data associated with the user health-related event. For example, the mobile device 104 may calculate an adherence ratio for the user and the health-related event. The adherence level may be determined based on the adherence ratio for the health-related event. The adherence ratio may be a quantitative measure of how frequently the user performs the health-related event in a given period of time (e.g., an adherence period). Stated differently, the adherence ratio may indicate the user's historical compliance with completion of the health-related event over the adherence period. The adherence ratio may be determined using the number of opportunities to perform the health-related event during the adherence period and the number of completions of the health-related event during the adherence period.

The mobile device 104 may determine to update the notification visibility level for the health-related event based on the determined adherence level. For example, the mobile device 104 may compare the adherence level for the health-related event to a plurality of predefined adherence thresholds. In response to comparing the adherence level to the plurality of predefined adherence thresholds, the mobile device 104 may be configured to determine a second notification visibility level for the user health-related event. The second notification visibility level may be determined to be a higher visibility level than the first notification visibility level when the adherence level is below a first predefined adherence threshold associated with the first notification visibility level. The second notification visibility level may be determined to be a lower visibility level than the first notification visibility level when the adherence level is greater than a second predefined adherence threshold associated with the first notification visibility level.

The mobile device 104 may increase the visibility of notifications associated with the health-related event when the adherence ratio (e.g., the frequency with which the user reports adherence to the health-related event) decreases. The mobile device 104 may decrease the visibility of notifications associated with the health-related event when the adherence ratio increases. Decreasing the visibility of notifications may be a reward for compliance with reporting adherence and/or an incentive to report adherence with other health-related events, for example, to reduce the number of notifications received. Receiving too many notifications for the behaviors/tasks can result in a poor user experience for the person with diabetes and, in some cases, a regression or desensitization from the importance of the behaviors/tasks. When a user receives too many notifications and/or alerts, the user tends to begin ignoring the notifications and/or alerts. Adjusting the visibility of notifications based on adherence may increase attentiveness to the notifications and/or alerts because unnecessary notifications and/or alerts are not sent and/or displayed to the user.

The mobile device 104 may be configured to group (e.g., bundle) two or more health-related events together in a notification group. Grouping the two or more health-related events may reduce the number of notifications sent and/or may increase compliance with adherence reporting for a low adherence event. For example, a health-related event with a low adherence ratio may be grouped with a health-related event with a high adherence ratio. Because the user regularly (e.g., with high adherence) completes the high adherence event, grouping the low adherence event with the high adherence event may trigger the user to perform the low adherence event when performing the high adherence event.

In some embodiments, the mobile device 104 and/or the remote computing device 122 may perform predictive learning. For example, the mobile device 104 and/or the remote computing device 122 may update a learning model of the user 100 as the user 100 performs a plurality of health-related events. The learning model may incorporate information from other users (e.g., users with similar characteristics) and/or historical data associated with the user to identify grouping targets for low adherence events and/or initial notification visibility levels. For example, the learning model may suggest one or more grouping targets for a low adherence event based on data showing how other users respond to grouping the low adherence event with the one or more grouping targets. In another example, the learning model may suggest an initial notification visibility level for a health-related event based on how other users comply with adherence reporting for the health-related event at various notification visibility levels. The learning model may receive data from the mobile device 104 and may update dynamically (e.g., as the user performs additional health-related events).

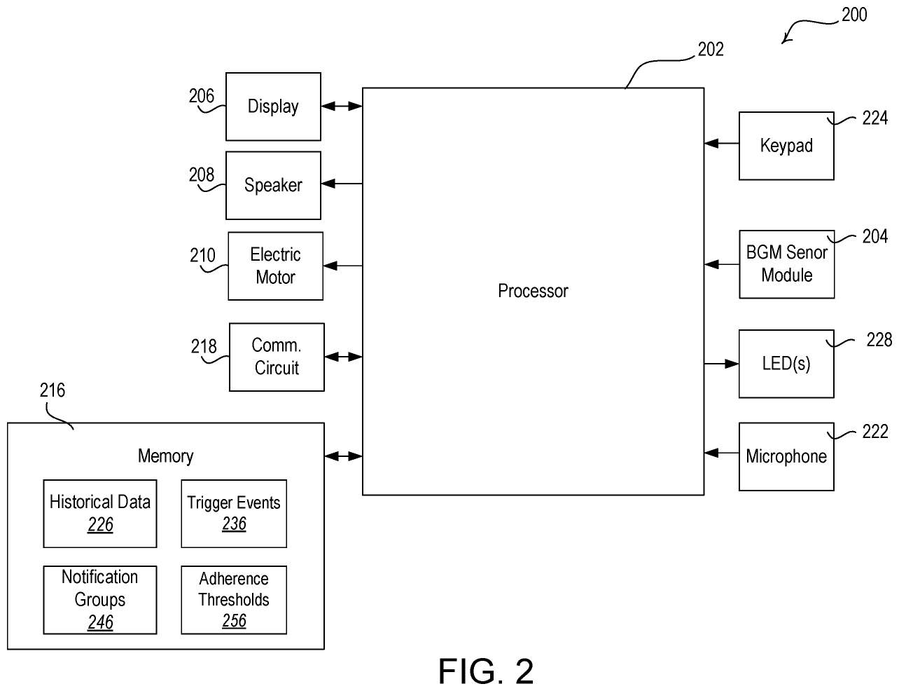

FIG. 2 is a block diagram of an example computing device 200. The computing device is a mobile computing device, such as a tablet, a cellular phone, a wearable device, a CGM controller device, or another computing device, for example. As shown in FIG. 2, the computing device 200 includes a processor 202 for controlling the functionality of the computing device 200. The processor 202 includes one or more circuits, such as general purpose processors, special purpose processors, conventional processors, digital signal processors (DSPs), microprocessors, integrated circuits, a programmable logic device (PLD), application specific integrated circuits (ASICs), or the like. The processor 202 performs signal coding, data processing, power control, image processing, input/output processing, or any other functionality that enables the computing device 200 to perform as described herein.

The processor 202 stores information in or retrieve information from the memory 216. The memory 216 includes a non-removable memory or a removable memory. The nonremovable memory may include random-access memory (RAM), read-only memory (ROM), a hard disk, or any other type of non-removable memory storage. The removable memory may include a subscriber identity module (SIM) card, a memory stick, a memory card (e.g., a digital camera memory card), or any other type of removable memory. The processor 202 accesses the memory 216 for executable instructions or other information that is used by the computing device 200. The memory 216 may store various information associated with one or more health-related events associated with a user of the computing device 200. For example, the memory 216 may be configured to store historical data 226 that is associated with the one or more health-related events, trigger events 236 associated with the one or more health-related events, notification groups 246 associated with the one or more health-related events, and/or adherence thresholds 256 associated with the one or more health-related events.

The computing device 200 includes a camera 206 that is in communication with the processor 202. The camera 206 is a digital camera or other optical device capable of generating images or videos (e.g., image sequences) for being captured at the computing device 200. The camera 206 includes a lighting device capable of flashing to in response to signals from the processor 202. The lighting device flashes to provide alerts via the camera 206.

The computing device 200 includes one or more communication circuits 218. The processor 202 is in electrical communication with the communication circuit 218 for sending or receiving information. The communication circuit 218 is capable of performing wired or wireless communications. For example, the communication circuit 218 includes one or more radio frequency (RF) transceivers for transmitting and receiving RF signals (e.g., BLUETOOTH®, near field communication (NFC), WIFI®, WI-MAX®, cellular, or other suitable wireless transceivers) via an antenna, or other communications module capable of performing wireless communications. One or more communication circuits 218 are capable of performing infrared (IR) communications.

The processor 202 is in electrical communication with a keypad 224 for providing input to the processor 202. The keypad 224 includes one or more keys for receiving input from a user. The keypad 224 includes hard or soft keys for which the function of the keys may change as a user performs selections.

Other input into the processor 202 is provided by one or more sensors 226. The sensors 226 includes a motion sensor, a proximity sensor, a heartrate monitoring sensor, an accelerometer, a gyroscope, or another sensor on the computing device. The motion sensor transmits infrared signals or use image processing to sense movement. The proximity sensor transmits infrared signals to detect when an object is within a predefined proximity. The heartrate monitoring sensor implements photoplethysmography to detect the amount of blood flow in the user. The heartrate monitoring sensor includes one or more LED or photodiodes to detect the amount of blood flow in the user. The heartrate monitoring sensor implements infrared technology to detect the amount of blood flow in the user. The heartrate monitoring sensor takes an electrocardiogram (ECG) and detects information about the user's heartrate from the ECG. The accelerometer measures the non-gravitational acceleration of the computing device 200 in a given direction. The accelerometer responds to vibrations associated with movement in a given direction. The measurements from the accelerometer are used by the processor 202 to determine the magnitude or direction of the relative movement of the computing device 200, or the user's relative position (e.g., standing, sitting, or lying down). The gyroscope is used to determine the orientation of the computing device 200.

The processor 202 is in electrical communication with or generate images on a display 220 for providing information to a user. The communication between the display 220 and the processor 202 is a two-way communication, as the display 220 includes a touch screen module capable of receiving information from a user and providing such information to the processor 202. For example, the display 220 provides soft buttons for selection by a user that are recognized by the touch screen module and provided to the processor 202 as input.

The processor 202 is in electrical communication with or control a speaker 208. The speaker 208 provides an audible sound (e.g., tone, beep, or buzz) in response to a triggering event detected by the processor 202.

The computing device 200 includes an electric motor 210 that is in electrical communication with or controlled by the processor 202. The computing device 200 may communicate a notification in response to detection of the triggering event. For example, the electric motor 210 rotates and causes the computing device 200 to vibrate (e.g., to indicate an alert) in response to a triggering event detected by the processor 202. The electric motor 210 provides an alert to supplement the audible alarm or replace the audible alarm provided by the speaker 208.

The processor 202 is in electrical communication with or receive information from a microphone 214. For example, the processor 202 receives audio signals via the microphone 214.

The computing device 200 includes a global positioning system (GPS) circuit 204. The GPS circuit 204 is capable of receiving GPS information. The processor 202 is capable of determining the GPS coordinates (e.g., latitude and longitude) of the computing device 200 based on the GPS information received via the GPS circuit.

The computing device 200 includes a visual indicator, such as one or more one or more light-emitting diodes (LEDs) 212. One or more LEDs 212 are illuminated or flashed to provide an alert or communicate other information to the user (e.g., low battery or turning on of the device).

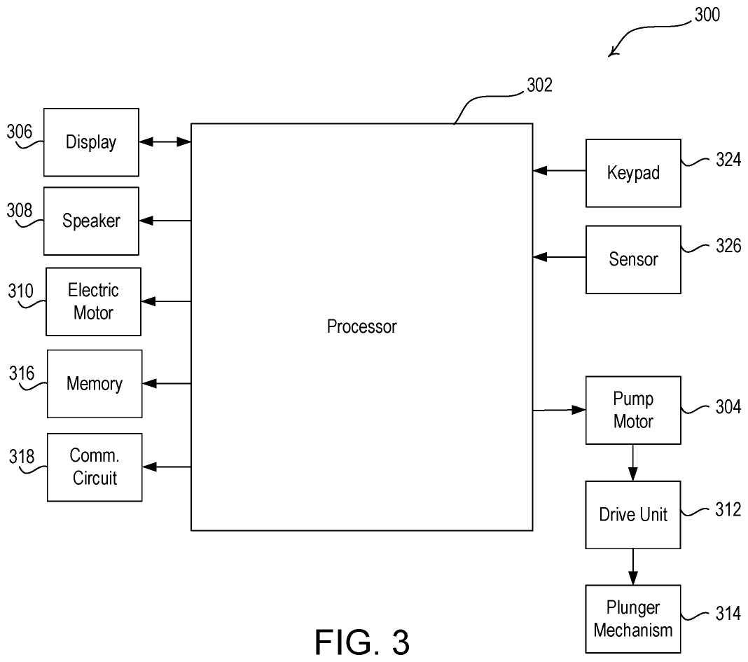

FIG. 3 is a block diagram of an example blood glucose monitoring device 300. The blood glucose monitoring device 300 is a CGM or FGM, for example. The blood glucose monitoring device 300 includes a subcutaneous sensor 326 that is used to sense and monitor the amount of glucose in interstitial fluid of the user. Data is transmitted from the sensor 326 to a transmitting device 304. When the blood glucose monitoring device 300 is a CGM, the transmitting device 304 is located directly over the sensor 326 and wirelessly powers the data transfer from the sensor 326 via power supply 320. When the blood glucose monitoring device 300 is an FGM, the transmitting device 304 is a mobile device or other reader device that instantaneously receives the blood glucose information from the sensor 326 when the device is within the RF range of the sensor 326.

The transmitting device 304 receives data communications from the sensor 326 via a communication circuit 318. The communication circuit 318 is in electrical communication with a processor 302. The processor 302 includes one or more circuits, such as general purpose processors, special purpose processors, conventional processors, digital signal processors (DSPs), microprocessors, integrated circuits, a programmable logic device (PLD), application specific integrated circuits (ASICs), or the like. The processor 302 performs signal coding, data processing, power control, input/output processing, or any other functionality that enables the transmitting device 304 to perform as described herein.

The transmitting device 304 includes another communication circuit 316 for communicating with other devices. The processor 302 is in electrical communication with the communication circuit 316 for sending or receiving information. The communication circuits 316, 318 are capable of performing wired or wireless communications. For example, the communication circuits 316, 318 includes one or more radio frequency (RF) transceivers for transmitting and receiving RF signals (e.g., BLUETOOTH®, near field communication (NFC), WIFI®, WI-MAX®, cellular, or other suitable RF signals) via an antenna, or other communications module capable of performing wireless communications. The communication circuits 316, 318 communicate using the same RF protocol or a different RF protocol.

The processor 302 stores information in or retrieves information from the memory 312. The memory 312 includes a non-removable memory or a removable memory. The nonremovable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The processor 302 accesses the memory 312 for executable instructions or other information that is used by the transmitting device 304. The processor 302 is in electrical communication with a one or more input keys 324 for providing input to the processor 302.

The processor 302 is in electrical communication with or controls a speaker 314. The speaker 314 provides an audible sound (e.g., tone, beep, or buzz) in response to a triggering event detected by the processor 302.

The blood glucose monitoring device 300 includes an electric motor 310 that is in electrical communication with or controlled by the processor 302. The blood glucose monitoring device 300 may communicate a notification in response to detection of the triggering event. For example, the electric motor 310 rotates and causes the blood glucose monitoring device 300 to vibrate (e.g., to indicate an alert) in response to a triggering event detected by the processor 302. The electric motor 310 provides an alert to supplement the audible alarm or replace the audible alarm provided by the speaker 314.

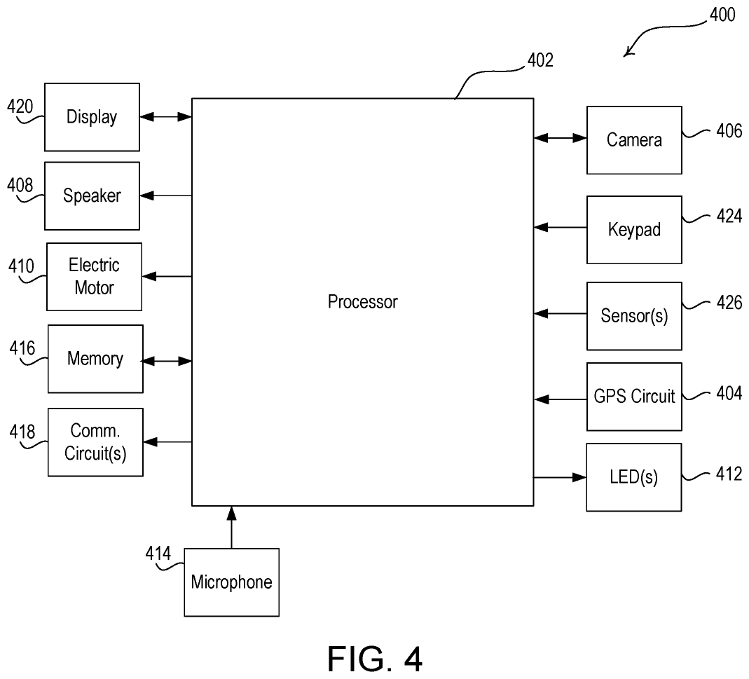

FIG. 4 is a block diagram of an example blood glucose measuring (BGM) device 400. As shown in FIG. 4, the BGM device 400 includes a processor 402 for controlling the functionality of the BGM device 400. In various embodiments, the processor 402 includes one or more digital logic devices, such as general-purpose processors, special purpose processors, digital signal processors (DSPs), microprocessors, integrated circuits, programmable logic devices (PLDs), application specific integrated circuits (ASICs), and any other suitable digital logic device. The processor 402 performs signal coding, data processing, power control, image processing, input/output processing, or any other functionality that enables the BGM device 400 to perform as described herein.

The processor 402 stores information in or retrieve information from the memory 416. The memory 416 includes a non-removable memory or a removable memory. The nonremovable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The processor 402 accesses the memory 416 for executable instructions or other information that is used by the BGM device 400.

The BGM device 400 includes one or more communication circuits 418. The processor 402 is in electrical communication with the communication circuit 418 for sending or receiving information. The communication circuit 418 is capable of performing wired or wireless communications. For example, the communication circuit 418 includes one or more radio frequency (RF) transceivers for transmitting and receiving RF signals (e.g., BLUETOOTH®, near field communication (NFC), WIFI®, WI-MAX®, cellular, or other suitable RF signals) via an antenna, or other communications module capable of performing wireless communications. One or more communication circuits 418 are capable of performing infrared (IR) communications.

The processor 402 is in electrical communication with a keypad 424 for providing input to the processor 402. The keypad 424 includes one or more keys for receiving input from a user. The keypad 424 includes hard or soft keys for which the function of the keys may change as a user performs selections.

Other input into the processor 402 is provided by the BGM sensor module 404. The BGM sensor module 404 includes a blood glucose measuring engine that analyzes blood samples provided by a patient on a blood glucose measurement strip and measures the amount of blood glucose in the samples.

The processor 402 is in electrical communication with or generate images on a display 406 for providing information to a user. The communication between the display 406 and the processor 402 is a two-way communication, as the display 406 includes a touch screen module capable of receiving information from a user and providing such information to the processor 402. For example, the display 406 provides soft buttons for selection by a user that are recognized by the touch screen module and provided to the processor 402 as input.

The processor 402 is in electrical communication with or control a speaker 408. The speaker 408 provides an audible sound (e.g., tone, beep, or buzz) in response to a triggering event detected by the processor 402.

The BGM device 400 includes an electric motor 410 that is in electrical communication with or controlled by the processor 402. The BGM device 400 may communicate a notification in response to detection of the triggering event. For example, the electric motor 410 rotates and causes the BGM device 400 to vibrate (e.g., to indicate an alert) in response to a triggering event detected by the processor 402. The electric motor 410 provides an alert to supplement the audible alarm or replace the audible alarm provided by the speaker 408.

The processor 402 is in electrical communication with or receive information from a microphone 422. For example, the processor 402 receives audio signals via the microphone 422.

The BGM device 400 includes a visual indicator, such as one or more one or more light emitting diodes (LEDs) 428. One or more LEDs 428 are illuminated or flashed to provide an alert or communicate other information to the user (e.g., low battery or turning on of the device).

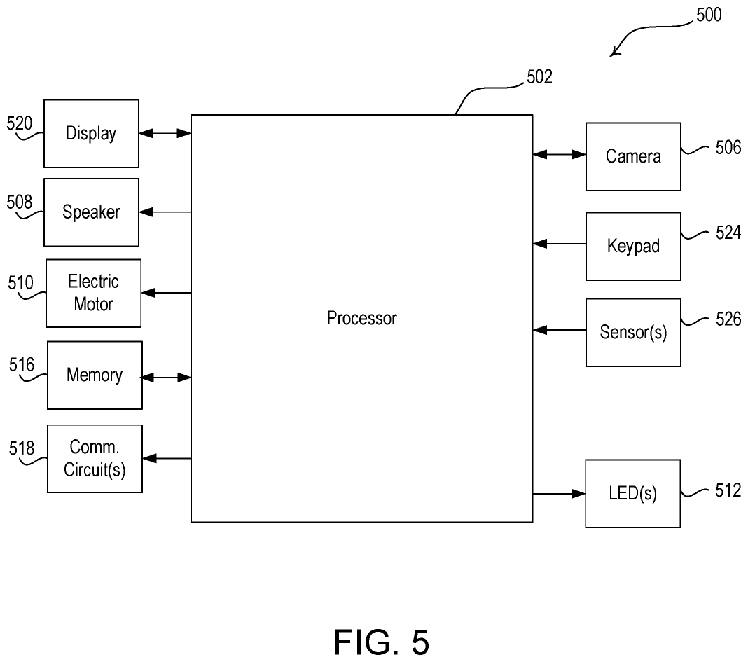

FIG. 5 is a block diagram illustrating an example of an insulin pump 500. As shown in FIG. 5, the insulin pump 500 includes a processor 502. The processor 502 includes one or more circuits, such as general-purpose processors, special purpose processors, conventional processors, digital signal processors (DSPs), microprocessors, integrated circuits, a programmable logic device (PLD), application specific integrated circuits (ASICs), or the like. The processor 502 performs signal coding, data processing, power control, image processing, input/output processing, or any other functionality that enables the insulin pump 500 to perform as described herein.

In the embodiment of FIG. 5, the processor 502 is in electrical communication with or control a pump motor 504 in the insulin pump 500. The pump motor 504 drives a drive unit 512 that pushes a plunger mechanism 514. The plunger mechanism 514 ejects insulin from an insulin cartridge (not shown). The insulin cartridge includes a supply of insulin for delivery to a user.

The processor 502 is in electrical communication with or generate images on a display 506 for providing information to a user. The communication between the display 506 and the processor 502 is a two-way communication, as the display 506 includes a touch screen module capable of receiving information from a user and providing such information to the processor 502. For example, the display 1306 provides soft buttons for selection by a user that are recognized by the touch screen module and provided to the processor 502 as input.

The processor 502 is in electrical communication with or control a speaker 508. The speaker 508 provides an audible sound (e.g., tone, beep, or buzz) in response to a triggering event detected by the processor 502.

The insulin pump 500 includes an electric motor 510 that is in electrical communication with or controlled by the processor 502. The insulin pump 500 may communicate a notification in response to detection of the triggering event. For example, the electric motor 510 rotates and causes the insulin pump to vibrate (e.g., to indicate an alert) in response to a triggering event detected by the processor 502. The electric motor 510 provides an alert to supplement the audible alarm or replace the audible alarm provided by the speaker 508.

The processor 502 is in electrical communication with a memory 516. The processor stores information in or retrieves information from the memory 516. The memory 516 includes a non-removable memory or a removable memory for storing computer-readable media. The non-removable memory includes random-access memory (RAM), read-only memory (ROM), a hard disk, or any other type of non-removable memory storage. The removable memory includes a subscriber identity module (SIM) card, a memory stick, a memory card (e.g., a digital camera memory card), or any other type of removable memory. The processor 502 accesses the memory 516 for executable instructions or other information that is used by the insulin pump 500.

The insulin pump 500 includes a communication circuit 518. The processor 502 is in electrical communication with the communication circuit 518 for sending or receiving information. The communication circuit 518 is capable of performing wired or wireless communications. For example, the wireless communications circuit 518 includes a radio frequency (RF) transceiver for transmitting and receiving RF signals (e.g., BLUETOOTH®, near field communication (NFC), WIFI®, WI-MAX®, cellular, or other suitable RF signals) via an antenna, or other communications module capable of performing wireless communications. The communication circuit 518 is capable of performing infrared (IR) communications.

The processor 502 is in electrical communication with a keypad 524 for providing input to the processor 502. The keypad 524 includes one or more keys for receiving input from a user. The keypad 524 includes hard or soft keys for which the function of the keys may change as a user performs selections.

Other input into the processor 502 is provided by sensors 526. The sensors 526 includes a pressure sensor that is sensitive to the pressure within a reservoir of insulin; a cartridge sensor that is sensitive to the presence of an insulin cartridge, or a motion sensor that detects the motion of a gear (not shown) in the drive unit 512.

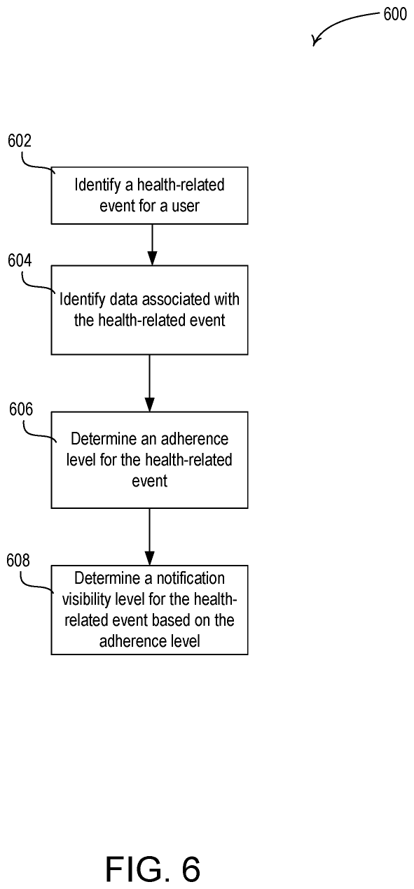

FIG. 6 shows a flowchart of an example process 600 for determining a notification visibility level of a health-related event on a computing device. The example process 600 is performed by one or more devices, such as one or more computing devices, for example. The process 600 is performed by a single computing device, or distributed across multiple devices. For example, the process 600 is performed by a computing device (e.g., such as mobile device 104 or remote computing device(s) 122 shown in FIG. 1). The process 600 may be performed by a health management application (e.g., such as mobile application 105) executed by the computing device. The example process 600 may be performed by a control circuit (e.g., such as the processor 502 shown in FIG. 5) executing instructions from a memory (e.g., such as the memory 516 shown in FIG. 5).

As illustrated in FIG. 6, a health-related event may be identified at 602. The health-related event may be a task (e.g., a new task or an existing task) assigned to a user. The health-related event may be associated with a health condition, disease, illness, and/or ailment. The health-related event may be a part of a structured testing period, part of a health management regimen, part of an exercise regimen, part of a personal hygiene regimen, part of a dietary program, and/or another health-related event. The health-related event may be identified at 602 in response to a user input. The user may input information associated with the health-related event into a health management application (e.g., such as the mobile application 105 shown in FIG. 1) of the computing device. For example, the user may select a “new task” or “create task” option in the mobile application to identify the health-related event. The health-related event may include consuming a meal, performing an activity (e.g., such as a type of exercise), taking a dose of medication, logging a medical reading (e.g., a blood glucose level, a body temperature, a heart rate, a blood pressure, an oxygen saturation level), logging sleep information, prescription status (e.g., prescription ordered, prescription filled, etc.), vaccination status, medical visits/checkups (e.g., dental visit, primary care visit, specialist visit, eye exam, etc.) and/or the like.

The computing device may identify, at 604, data associated with the health-related event. The data associated with the health-related event may be historical data and/or real-time data. For example, the computing device may query a database (e.g., such as datastore(s) 124 shown in FIG. 1) for historical data associated with the health-related event. The database may be a local database (e.g., on the computing device) or a remote database (e.g., on a remote computing device). The remote database may be located in the cloud and the computing device may access the remote database via a network (e.g., If historical data associated with the health-related event is located in the database, the computing device may link the data to the health-related event. The historical data located in the database may be historical data associated with the health-related event and/or another health-related event that is similar or related to the health-related event. The computing device may identify, at 604, real-time data based on data received from the computing devices. For example, the computing device (e.g., the health management application) may link a blood glucose reading from a blood glucose monitor (e.g., the CGM 102 or the BGM 106 shown in FIG. 1) to the health-related event. The computing device (e.g., the health management application) may link medication data for a medication and/or dietary data for a meal to the health-related event based on a scan of a unique identifier associated with medication and/or meal. For example, at least a portion of the historical and/or real-time data may be received by the computing device upon scanning one or more of a bar code, a quick response (QR) code, a radio frequency identification (RFID) tag, or a near field communication (NFC) tag attached to the medication or meal. The computing device may store the real-time data in the database.

The data may include one or more blood glucose levels, a dietary entry, a medication entry, an exercise entry, a body temperature, a heart rate, a blood pressure, an oxygen saturation level, sleep information, lab data, manual data entry, and/or the like. In examples, the data may include a manual entry indicating completion of the health-related event. The manual entry may include a user response to a survey. In examples, the data may include an automated entry indicating completion of the health-related event. For example, the computing device may determine that the health-related event has been completed based on the data identified, at 604.

The computing device may determine, at 606, an adherence level for the health-related event. The adherence level may be calculated based on an adherence ratio. The adherence ratio may be determined, at 606, based on the data associated with the health-related event. The adherence ratio may be a quantitative measure of how frequently the user performs the health-related event in a given period of time (e.g., an adherence period). Stated differently, the adherence ratio may indicate the user's historical compliance with completion of the health-related event over the adherence period. The adherence ratio may be determined, at 606, using the number of opportunities to perform the health-related event during the adherence period and the number of completions of the health-related event during the adherence period.

The adherence period (e.g., a duration of the adherence period) may be predetermined and may be based on the health-related event. For example, different health-related events may have different adherence periods for adherence level determination. The health-related event may be a task that the user is required to perform on a regular schedule (e.g., daily, weekly, etc.). The adherence period may include the most recent period of time (e.g., 30 days, 60 days, 90 days, etc.) prior to the adherence level determination. Moreover, the adherence period may be adjusted over time, for example, based on the adherence level. For example, newer tasks (e.g., less than 7 days, 14 days, or 30 days old) may be associated with shorter adherence periods than older tasks. Stated differently, as a task is performed by the user over time, the duration of the adherence period for that task may increase.

The adherence level may indicate a probability that the user will perform a given instance of the health-related event. The computing device (e.g., the health management application) may use a predictive model (e.g., using predictive learning) to determine the probability that the user will perform the given instance of the health-related event. For example, the predictive model may determine the probability using historical and/or real-time data (e.g., adherence data) associated with one or more similar health-related events.

The adherence level may include a range of adherence for the user-related event. The user-related event may be associated with a plurality of adherence levels. Each of the plurality of adherence levels may be defined by an upper adherence threshold and a lower adherence threshold, respectively. The range of adherence may include a plurality of adherence levels between the upper adherence threshold and the lower adherence threshold. An example set of adherence levels is shown in Table 1.

TABLE 1
Example Adherence Levels

| Adherence Level | Adherence Ratio |
|---|---|
| Level 1 | <20% |
| Level 2 | 20-49% |
| Level 3 | 50-79% |
| Level 4 | ≥80% |
| Undefined | Cannot be |
| | evaluated |

In the example set of adherence levels shown in Table 1, adherence level 1 is defined by a lower adherence threshold of 0% and an upper adherence threshold of 20%, adherence level 2 is defined by a lower adherence threshold of 20% and an upper adherence threshold of 49%, adherence level 3 is defined by a lower adherence threshold of 50% and an upper adherence threshold of 79%, and adherence level 4 is defined by a lower adherence threshold of 80% and an upper adherence threshold of 100%.

The plurality of adherence levels may include an undefined adherence level for which the adherence level is unable to be determined. The communication status of the computing device (e.g., mobile health application) may be checked regularly (e.g., daily). The computing device may record instances of successful communication such that the computing device can differentiate between loss of communication and a failure to log data/report compliance. When the computing device is in a loss of communication status for more than a predetermined number of days during the adherence period, the computing device may select the undefined adherence level. The number of days in which the computing device is in a loss of communication status may be used to determine the adherence level. For example, the instances where adherence was not reported that correspond to times when the computing device was in a loss of communication status may be omitted from the adherence level determination. Using the loss of communication status in the adherence level determination may prevent misinterpreting loss of communication as failed compliance with the health-related event.

The plurality of adherence levels may be adjustable based on one or more of a characteristic of the user, an age of the health-related event, a characteristic of the health-related event, a number of times adherence of the health-related event has been logged within a predefined time period, or a frequency of the health-related event. One or more of the upper adherence thresholds and/or lower adherence thresholds may be adjusted for a specific health-related event. For example, the lower adherence threshold for one or more levels of a critically important health-related event may be increased according to the relative importance of the health-related event such that a greater adherence percentage is required for the one or more levels. The health-related event may be critically important to the user and/or to a third party. For example, the adherence levels for critically important health-related events may include higher lower adherence thresholds and/or smaller adherence ranges for one or more adherence levels. Additionally or alternatively, the adherence levels may be adjustable as a health related event becomes a frequently performed task. For example, the lower adherence thresholds may be reduced as the health-related event becomes a frequently performed task.

Additionally or alternatively, the adherence level may be determined, at 606, based on data associated with other users (e.g., other users having similar traits and/or characteristics) associated with the health-related event. For example, data associated with other user associated with the health-related event may be used to determine an initial adherence level. The computing device (e.g., mobile health application) may identify one or more (e.g., a group of) users having a similar age, gender, activity level, medical history, etc. The computing device (e.g., mobile health application) may aggregate adherence data of the one or more users for the health-related event to determine the adherence level for the user. For example, the computing device (e.g., mobile health application) may generate and/or maintain an adherence level model. The adherence level model may be used to determine an adherence level for a specific health-related event for a user within an age range, having a specific gender, having a specific activity level, and/or specific medical history.

The computing device may determine, at 608, a notification visibility level for the health-related event based on the adherence level determined at 606. The notification visibility level may be a measure of the visibility to the user of a notification associated with the user health-related event. For example, health-related events with a lower adherence level may be assigned a higher notification visibility level than health-related events with a higher adherence level. Having different notification visibility levels may incentivize the user to do a good job adhering to their health-related events. For example, the user may want to reduce the number of notifications/alerts received.

The notification visibility level may be determined, at 608, based on an age associated with the health-related event. For example, newer health-related events may be assigned a higher notification visibility level when compared to older health-related events having the same or similar adherence levels. The notification visibility level may be determined, at 608, using a notification visibility model that calculates the notification visibility level for a health-related event using the adherence level associated with the health-related event and the length of time that the user has been performing the health-related event.

The notification visibility level may be determined, at 608, from a plurality of notification visibility levels that include an intervention visibility level, a request input visibility level, a dashboard visibility level, and a hidden visibility level. The intervention visibility level may be the highest notification visibility level of the plurality of notification visibility levels. When the intervention visibility level is assigned to a health-related event, a notification may be sent to the user (e.g., the computing device) and a notification may be sent to another user (e.g., a trusted care partner, a coach, a care coordinator, a nurse, a doctor, etc.) who may be able to provide motivation to perform the health-related event. When the request input visibility level is assigned to a health-related event, the computing device (e.g., mobile health application) may display a notification and/or reminder to log completion of the health-related event. For example, the notification and/or reminder may include an embedded link that forwards the user to a screen where the user can log completion of the health-related event.

When the dashboard visibility level is assigned to a health-related event, the computing device (e.g., mobile health application) may include the health-related event in a list of tasks to be completed in a given time period (e.g., daily, weekly, monthly, quarterly, etc.). In examples, the dashboard visibility level may be configured to display the list of tasks to be completed for a given day, for example, to simplify the view and avoid overwhelming the user with too many tasks. The list of tasks may be referred to as a to-do list, a dashboard, and/or the like. In examples, the dashboard visibility level may enable (e.g., via a calendar widget) display of the list of tasks within a calendar application on the computing device. In this case, the calendar application may display a minimized version of the list that prioritizes tasks that are expected within the next portion of the day (e.g., predetermined number of hours) and/or tasks that match a current context (e.g., location and time of day). When the hidden visibility level is assigned to a health-related event, the computing device (e.g., health management application) may be hidden from view (e.g., not displayed to the user). For example, the hidden health-related event may not be displayed in notifications and/or a list of tasks to be completed. Adherence data for hidden health-related events may be accessed via the health management application.

The notification visibility level may be determined, at 608, dynamically. For example, the notification visibility level may be updated (e.g., daily, weekly, etc.) as the user performs the health-related event over time. The notification visibility level may be updated using data from the most recent period of time (e.g., 7 days, 14 days, 30 days, etc.). When the user's adherence to the health-related event increases (e.g., above an adherence threshold), the notification visibility level may be decreased such that the user is not notified when the health-related event is scheduled to be completed. The computing device (e.g., mobile health application) may display a notification to the user when the notification visibility level of a health-related event changes. For example, the notification may indicate that the notification visibility level change is a reward for maintaining adherence level above a predetermined threshold. When a user sees that he/she will not be receiving notifications for a health-related event due to a high adherence to the health-related event, the user may be encouraged/motivated to increase adherence to other health-related events to decrease the number of notifications and/or alerts received.

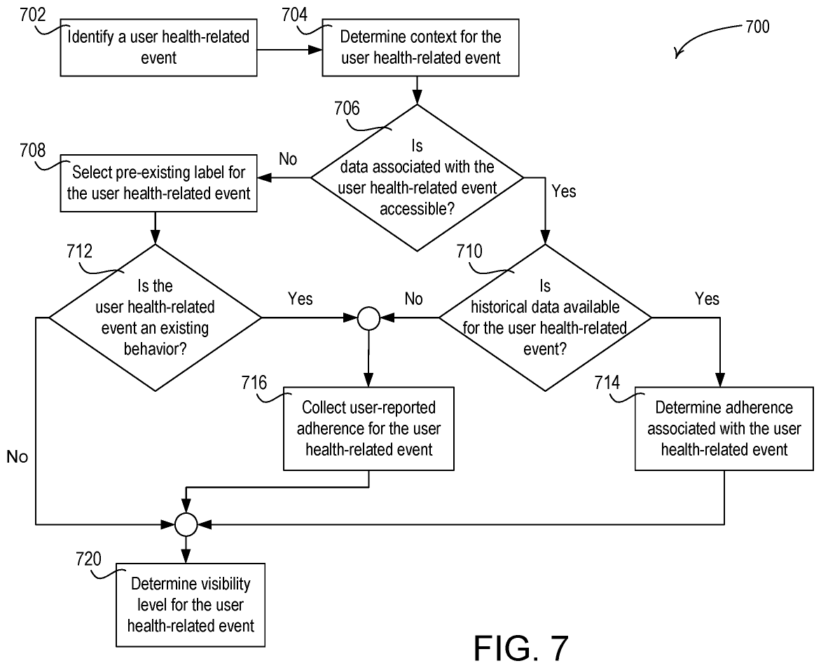

FIG. 7 shows a flowchart of another example process 700 for determining a notification visibility level of a health-related event on a computing device. The example process 700 is performed by one or more devices, such as one or more computing devices, for example. The process 700 may be performed by a single computing device, or may be distributed across multiple devices. For example, the process 700 may be performed by a computing device (e.g., such as mobile device 104 or remote computing device(s) 122 shown in FIG. 1). The process 700 may be performed by a health management application (e.g., such as mobile application 105) executed by the computing device. The example process 700 may be performed by a control circuit (e.g., such as the processor 502 shown in FIG. 5) executing instructions from a memory (e.g., such as the memory 516 shown in FIG. 5).

As illustrated in FIG. 7, a health-related event may be identified at 702. The health-related event may be a task (e.g., a new task or an existing task) assigned to a user. For example, the health-related event may be a task that the user is required to perform on a regular schedule (e.g., daily, weekly, etc.). The health-related event may be associated with a health condition, disease, illness, and/or ailment. The health-related event may be a part of a structured testing period, part of a health management regimen, part of an exercise regimen, part of a personal hygiene regimen, part of a dietary program, and/or another health-related event. The health-related event may be identified at 702 in response to a user input. The user may input information associated with the health-related event into a health management application (e.g., such as the mobile application 105 shown in FIG. 1) of the computing device. For example, the user may select a “new task” or “create task” option in the mobile application to identify the health-related event. The health-related event may include consuming a meal, performing an activity (e.g., such as a type of exercise), taking a dose of medication, logging a medical reading (e.g., a blood glucose level, a body temperature, a heart rate, a blood pressure, an oxygen saturation level), logging sleep information, prescription status (e.g., prescription ordered, prescription filled, etc.), vaccination status, medical visits/checkups (e.g., dental visit, primary care visit, specialist visit, eye exam, etc.) and/or the like.

The computing device may determine, at 704, a context associated with the health-related event. The context may include a time, a location, one or more user characteristics and/or one or more event characteristics. The one or more user characteristics may include education level, socio-economic status, age, gender, marital status, and/or the like. The one or more event characteristics may include an activity code assigned to the health-related event. In examples, each health-related event may be assigned a unique activity code. In other examples, a group of similar health-related events may be assigned a group activity code.

The computing device may determine, at 706, whether data associated with the health-related event is accessible. For example, the computing device may query its memory (e.g., such as memory 216 shown in FIG. 2) to identify any data associated with the health-related event. The computing device may also query an external database (e.g., such as datastore(s) 124 shown in FIG. 1) to identify any data associated with the health-related event. Data associated with the health-related event may not be accessible when the health-related event is new to the user and/or system.

When no data associated with the health-related event is accessible, the user device may select, at 708, a pre-existing label for the health-related event. Additionally or alternatively, the user may input a label for the health-related event. For example, the user may input information associated with the health-related event into the health management application of the user device to be used as the label for the health-related event.

The user device may determine, at 712, whether the health-related event is an existing behavior. When the health-related event is not an existing behavior, the user device may determine, at 720, a visibility level for the health-related event based on data associated with other users who have performed the health-related event. The user device may identify one or more (e.g., a group of) users having a similar age, gender, activity level, medical history, etc. The user device may aggregate adherence data of the one or more users for the health-related event to determine an initial visibility level for the user. For example, the user device may generate and/or maintain an adherence level model. The adherence level model may be used to determine the initial visibility level for a specific health-related event for a user within an age range, having a specific gender, having a specific activity level, and/or specific medical history. Additionally or alternatively, when the health-related event is not an existing behavior, the user device may determine, at 720, an initial visibility level for the health-related event. The initial visibility level may be a request input visibility level. When the request input visibility level is assigned to the health-related event, the computing device may display a notification and/or reminder to log completion of the health-related event. For example, the notification and/or reminder for the request input visibility level may include an embedded link that forwards the user to a screen where the user can log completion of the health-related event.

When the health-related event is an existing behavior, the computing device may collect, at 716, user-reported adherence for the health-related event. The user-reported adherence may include a manual entry indicating completion of the health-related event. The manual entry may include a user response to a survey. In examples, the data may include an automated entry indicating completion of the health-related event. For example, the computing device may determine that the health-related event has been completed based on the data collected. Additionally or alternatively, the collected user-reported adherence may include real-time data based on data received from other computing devices. For example, the computing device may link a blood glucose reading from a blood glucose monitor (e.g., the CGM 102 or the BGM 106 shown in FIG. 1) to the health-related event. The computing device (e.g., the health management application) may link medication data for a medication and/or dietary data for a meal to the health-related event based on a scan of a unique identifier associated with a medication and/or a meal. For example, at least a portion of the user-reported adherence data may be received by the computing device upon scanning one or more of a bar code, a quick response (QR) code, a radio frequency identification (RFID) tag, or a near field communication (NFC) tag attached to the medication or meal. The computing device may log the user-reported adherence data, for example, in the database.

When data associated with the health-related event is accessible, the computing device may determine, at 710, whether historical data is available for the user health-related event. For example, the computing device may query a database (e.g., such as datastore(s) 124 shown in FIG. 1) and/or a remote computing device (e.g., such as remote computing device 122 shown in FIG. 1) for historical data associated with the health-related event. The database may be a local database (e.g., on the computing device) or a remote database (e.g., on a remote computing device). The remote database may be located in the cloud and the computing device may access the remote database via a network (e.g., such as the network 120 shown in FIG. 1). If historical data associated with the health-related event is located in the database, the computing device may link the data to the health-related event. The historical data located in the database may be historical data associated with the health-related event and/or another health-related event that is similar or related to the health-related event. When no historical data is available for the user health-related event, the method may proceed to 716.

When historical data is available for the user health-related event, the computing device may determine, at 714, an adherence level associated with the health-related event. For example, the computing device may use the historical data to determine, at 714, the adherence level. The method may proceed to 720 such that the computing device may determine, at 720, the visibility level for the health-related event using the adherence level determined at 714.

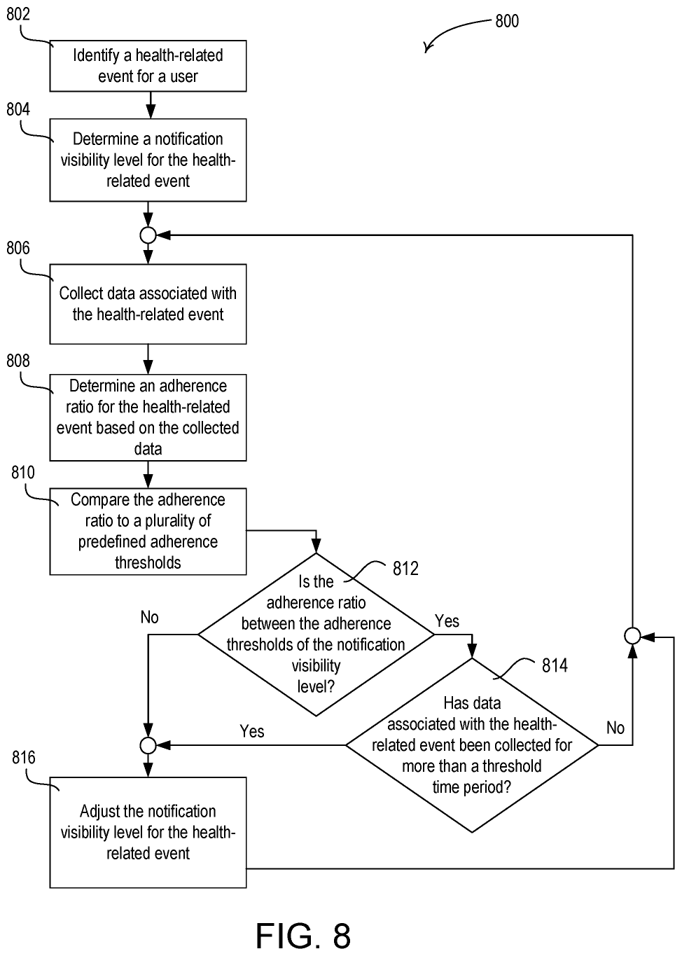

FIG. 8 shows a flowchart of an example process 800 for adjusting a notification visibility level of a health-related event on a computing device. The example process 800 is performed by one or more devices, such as one or more computing devices, for example. The process 800 is performed by a single computing device, or distributed across multiple devices. For example, the process 800 is performed by a computing device (e.g., such as mobile device 104 or remote computing device(s) 122 shown in FIG. 1). The process 800 may be performed by a health management application (e.g., such as mobile application 105) executed by the computing device. The example process 800 may be performed by a control circuit (e.g., such as the processor 502 shown in FIG. 5) executing instructions from a memory (e.g., such as the memory 516 shown in FIG. 5).

As illustrated in FIG. 8, a health-related event may be identified at 802. The health-related event may be a task (e.g., a new task or an existing task) assigned to a user. For example, the health-related event may be a task that the user is required to perform on a regular schedule (e.g., daily, weekly, etc.). The health-related event may be associated with a health condition, disease, illness, and/or ailment. The health-related event may be a part of a structured testing period, part of a health management regimen, part of an exercise regimen, part of a personal hygiene regimen, part of a dietary program, and/or another health-related event. The health-related event may be identified at 802 in response to a user input. The user may input information associated with the health-related event into a health management application (e.g., such as the mobile application 105 shown in FIG. 1) of the computing device. For example, the user may select a “new task” or “create task” option in the mobile application to identify the health-related event. The health-related event may include consuming a meal, performing an activity (e.g., such as a type of exercise), taking a dose of medication, logging a medical reading (e.g., a blood glucose level, a body temperature, a heart rate, a blood pressure, an oxygen saturation level), logging sleep information, prescription status (e.g., prescription ordered, prescription filled, etc.), vaccination status, medical visits/checkups (e.g., dental visit, primary care visit, specialist visit, eye exam, etc.) and/or the like.

The computing device may determine, at 804, a notification visibility level for the health-related event identified at 802. The computing device may determine, at 804, a visibility level for the health-related event based on data associated with other users who have performed the health-related event. The computing device may identify one or more (e.g., a group of) users having a similar age, gender, activity level, medical history, etc. The computing device may aggregate adherence data of the one or more users for the health-related event to determine an initial visibility level for the user. For example, the computing device may generate and/or maintain an adherence level model. The adherence level model may be used to determine the initial visibility level for a specific health-related event for a user within an age range, having a specific gender, having a specific activity level, and/or specific medical history.

Additionally or alternatively, when the health-related event is not an existing behavior, the computing device may determine, at 804, an initial visibility level for the health-related event. The initial visibility level may be a request input visibility level. When the request input visibility level is assigned to the health-related event, the computing device may display a notification and/or reminder to log completion of the health-related event. For example, the notification and/or reminder for the request input visibility level may include an embedded link that forwards the user to a screen where the user can log completion of the health-related event.

The computing device may collect, at 806, data associated with the health-related event. The data may include a manual entry indicating completion of the health-related event. The manual entry may include a user response to a survey. In examples, the data may include an automated entry indicating completion of the health-related event. For example, the computing device may determine that the health-related event has been completed based on the data collected. Additionally or alternatively, the data collected, at 806, may include real-time data based on data received from other computing devices. For example, the computing device may link a blood glucose reading from a blood glucose monitor (e.g., the CGM 102 or the BGM 106 shown in FIG. 1) to the health-related event. The computing device may link medication data for a medication and/or dietary data for a meal to the health-related event based on a scan of a unique identifier associated with a medication and/or a meal. For example, at least a portion of the data may be received by the computing device upon scanning one or more of a bar code, a quick response (QR) code, a radio frequency identification (RFID) tag, or a near field communication (NFC) tag attached to the medication or meal. The computing device may log the data associated with the health-related event, for example, in the database.

The data may include one or more blood glucose levels, a dietary entry, a medication entry, an exercise entry, a body temperature, a heart rate, a blood pressure, an oxygen saturation level, sleep information, lab data, manual data entry, and/or the like. In examples, the data may include a manual entry indicating completion of the health-related event. The manual entry may include a user response to a survey. In examples, the data may include an automated entry indicating completion of the health-related event. For example, the computing device may determine that the health-related event has been completed based on the data collected, at 806.

The computing device may determine, at 808, an adherence ratio for the health-related event. The adherence ratio may be determined, at 606, based on the data associated with the health-related event. The adherence ratio may be a quantitative measure of how frequently the user performs the health-related event in a given period of time (e.g., an adherence period). Stated differently, the adherence ratio may indicate the user's historical compliance with completion of the health-related event over the adherence period. The adherence ratio may be determined, at 808, using the number of opportunities to perform the health-related event during the adherence period and the number of completions of the health-related event during the adherence period.

The adherence period (e.g., a duration of the adherence period) may be predetermined and may be based on the health-related event. For example, different health-related events may have different adherence periods for adherence level determination. The adherence period may include the most recent period of time (e.g., 30 days, 60 days, 90 days, etc.) prior to the adherence level determination. Moreover, the adherence period may be adjusted over time, for example, based on the adherence level. For example, newer tasks (e.g., less than 7 days, 14 days, or 30 days old) may be associated with shorter adherence periods than older tasks. Stated differently, as a task is performed by the user over time, the duration of the adherence period for that task may increase.

The computing device may collect, at 806, data associated with the health-related event. The data associated with the health-related event may include data that is manually entered by the user to indicate completion of the health-related event. The manual entry may include a user response to a survey. In examples, the data may include an automated entry indicating completion of the health-related event. For example, the computing device may determine that the health-related event has been completed based on the data collected. Additionally or alternatively, the data associated with the health-related event may include real-time data based on data received from other computing devices. For example, the computing device may link a blood glucose reading from a blood glucose monitor (e.g., the CGM 102 or the BGM 106 shown in FIG. 1) to the health-related event. The computing device may link medication data for a medication and/or dietary data for a meal to the health-related event based on a scan of a unique identifier associated with a medication and/or a meal. For example, at least a portion of the data may be received by the computing device upon scanning one or more of a bar code, a quick response (QR) code, a radio frequency identification (RFID) tag, or a near field communication (NFC) tag attached to the medication or meal. The computing device may log the collected data, for example, in the database.

The data associated with the health-related event may include one or more blood glucose levels, a dietary entry, a medication entry, an exercise entry, a body temperature, a heart rate, a blood pressure, an oxygen saturation level, sleep information, lab data, manual data entry, and/or the like. In examples, the data may include a manual entry indicating completion of the health-related event. The manual entry may include a user response to a survey. In examples, the data may include an automated entry indicating completion of the health-related event. For example, the computing device may determine that the health-related event has been completed based on the data collected, at 806.

The computing device may determine, at 808, an adherence ratio for the health-related event. The adherence ratio may be determined, at 808, based on the collected data associated with the health-related event. The adherence ratio may be a quantitative measure of how frequently the user performs the health-related event in a given period of time (e.g., an adherence period). Stated differently, the adherence ratio may indicate the user's historical compliance with completion of the health-related event over the adherence period. The adherence ratio may be determined, at 808, using the number of opportunities to perform the health-related event during the adherence period and the number of completions of the health-related event during the adherence period. The completions of the health-related event may be determined based on the collected data.

The adherence period (e.g., a duration of the adherence period) may be predetermined and may be based on the health-related event. For example, different health-related events may have different adherence periods for adherence level determination. The health-related event may be a task that the user is required to perform on a regular schedule (e.g., daily, weekly, etc.). The adherence period may include the most recent period of time (e.g., 30 days, 60 days, 90 days, etc.) prior to the adherence level determination. Moreover, the adherence period may be adjusted over time, for example, based on the adherence level. For example, newer tasks (e.g., less than 7 days, 14 days, or 30 days old) may be associated with shorter adherence periods than older tasks. Stated differently, as a task is performed by the user over time, the duration of the adherence period for that task may increase.

The computing device may compare, at 810, the adherence ratio to a plurality of predefined adherence thresholds. The plurality of predefined adherence thresholds may be adherence ratios that define a plurality of adherence levels. For example, respective predefined adherence thresholds may define an upper adherence threshold and lower adherence threshold for one of the adherence levels.

The computing device may determine, at 812, whether the adherence ratio is between adherence thresholds associated with a current notification visibility level (e.g., the notification visibility level determined at 804). For example, the current notification visibility level may be associated with an adherence level of the plurality of adherence levels. The computing device may determine, at 812, whether the adherence ratio of the user for the health-related event remains within the adherence thresholds of the adherence level associated with the current notification visibility level.

When the adherence ratio is within the adherence thresholds of an adherence level associated with the current notification visibility level, the computing device may determine, at 814, whether data associated with the health-related event has been collected for greater than a threshold time period. The threshold time period may be a time period associated with the current notification visibility level. The threshold time period may represent an amount of time between classifying a new habit as a short-term habit and/or an amount of time between classifying a short-term habit as an established habit. For example, a health-related event may be classified as a new habit when it has been performed by the user for less than a short-term habit threshold (e.g., one week, one month, and/or the like). The health-related event may be classified as a short-term habit when it has been performed longer than the short-term habit threshold but less than an established habit threshold (e.g., one month, two months, six months, and/or the like). The health-related event may be classified as an established habit when it has been performed longer than the established habit threshold. Each of the threshold time periods may be associated with a notification visibility level. For example, the notification visibility level of a health-related event with a high adherence level (e.g., >80% adherence ratio) may transition from a request input visibility level when classified as a new habit, to a dashboard visibility level when classified as a short-term habit, and to a hidden visibility level when classified as an established habit. The computing device may determine, at 814, if the upper threshold time period for the current notification visibility level has been exceeded.

When the upper threshold time period for the current notification visibility level has not been exceeded, the computing device may proceed to 806 to continue collecting (e.g., and logging) data associated with the health-related event.

When the upper threshold time period for the current notification visibility level has been exceeded, the computing device may adjust, at 816, the notification visibility level for the health-related event. For example, the computing device may update the notification visibility level based on the amount of time that the user has performed the health-related event. The current notification visibility level may be adjusted to an updated notification visibility level based on the upper threshold time period being exceeded. For example, when the current notification visibility level of the health-related event is at a request input visibility level and the upper threshold time period for a new habit/short-term habit has been exceeded, the notification visibility level may be adjusted to the dashboard visibility level. When the notification visibility level has been adjusted, the computing device may proceed to 806 to continue collecting (e.g., and logging) data associated with the health-related event.

When the adherence ratio is outside of the adherence thresholds of an adherence level associated with the current notification visibility level, the computing device may adjust, at 816, the notification visibility level for the health-related event, as described herein.

The process 800 may be regularly performed by the computing device. For example, the computing device may perform the process 800 daily (e.g., at the end of each day), weekly, biweekly, and/or monthly.

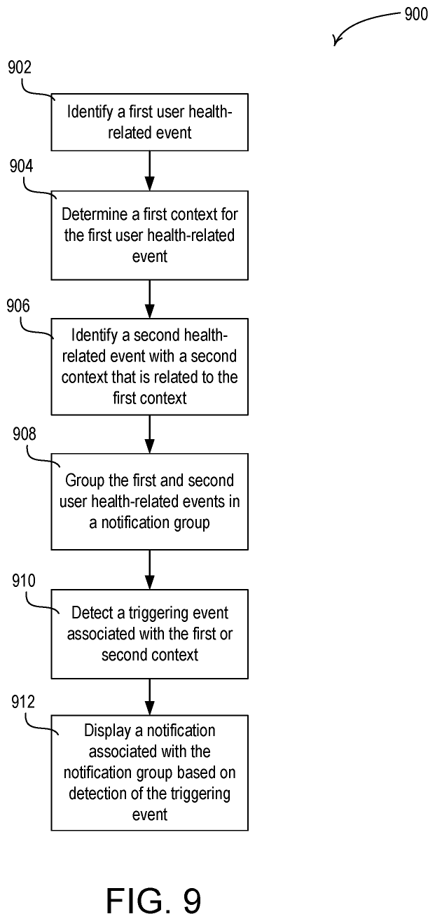

FIG. 9 shows a flowchart of another example process 900 for grouping health-related events into a notification group. The example process 900 is performed by one or more devices, such as one or more computing devices, for example. The process 900 may be performed by a single computing device, or may be distributed across multiple devices. For example, the process 900 may be performed by a computing device (e.g., such as mobile device 104 or remote computing device(s) 122 shown in FIG. 1). The process 900 may be performed by a health management application (e.g., such as mobile application 105) executed by the computing device. The example process 900 may be performed by a control circuit (e.g., such as the processor 502 shown in FIG. 5) executing instructions from a memory (e.g., such as the memory 516 shown in FIG. 5). Grouping health-related events into notification groups may reduce the number of notifications and/or interactions required by the user. For example, the computing device may display one notification for the notification group instead of one notification for each health-related event. In examples, the computing device may display time of day notifications (e.g., morning, afternoon, evening, bedtime, etc.) by notification group (e.g., morning notification group, afternoon notification group, evening notification group, bedtime notification group, etc.).

As illustrated in FIG. 9, a health-related event may be identified at 902. The health-related event may be a task (e.g., a new task or an existing task) assigned to a user. For example, the health-related event may be a task that the user is required to perform on a regular schedule (e.g., daily, weekly, etc.). The health-related event may be associated with a health condition, disease, illness, and/or ailment. The health-related event may be a part of a structured testing period, part of a health management regimen, part of an exercise regimen, part of a personal hygiene regimen, part of a dietary program, and/or another health-related event. The health-related event may be identified at 902 in response to a user input. The user may input information associated with the health-related event into a health management application (e.g., such as the mobile application 105 shown in FIG. 1) of the computing device. For example, the user may select a “new task” or “create task” option in the mobile application to identify the health-related event. The health-related event may include consuming a meal, performing an activity (e.g., such as a type of exercise), taking a dose of medication, logging a medical reading (e.g., a blood glucose level, a body temperature, a heart rate, a blood pressure, an oxygen saturation level), logging sleep information, prescription status (e.g., prescription ordered, prescription filled, etc.), vaccination status, medical visits/checkups (e.g., dental visit, primary care visit, specialist visit, eye exam, etc.) and/or the like.

The computing device may determine, at 904, a first context associated with the health-related event. The first context may include a time, a location, one or more user characteristics and/or one or more event characteristics. The one or more user characteristics may include education level, socio-economic status, age, gender, marital status, and/or the like. The one or more event characteristics may include an activity code assigned to the health-related event. In examples, each health-related event may be assigned a unique activity code. In other examples, a group of similar health-related events may be assigned a group activity code.

The computing device may identify, at 906, a second health-related event with a second context that is related to the first context. In examples, the second context may be the same as the first context. In other examples, the second context may be similar to the first context. When the first context is a first time of day, the second context may be a second time of day that is near the first time of day. For example, the first context may be 9:00 am and the second context may be morning. Because 9:00 am is in the morning, the second context may be considered as related to the first context. Additionally or alternatively, the first context may include a location (e.g., such as home, school, work, restaurant, other). When the second context matches the location, the second health-related event may be identified, at 906.

The computing device may group, at 908, the first and second health-related events into a notification group. For example, the first and second health-related events may be grouped into the notification group based on having similar contexts. Grouping health-related events into notification groups may reduce the number of notifications and/or interactions required by the user. For example, the computing device may display one notification for the notification group instead of one notification for each health-related event. Additionally or alternatively, grouping health-related events may increase adherence with one or more health-related events. For example, the second health-related event may be a habit having a high adherence ratio (e.g., greater than 80%). The high adherence ratio habit may be in a hidden notification visibility level and/or is performed by the user without being monitored or logged. Grouping the first health-related event with the high adherence ratio habit may make it easier for the user to remember to perform the first health-related event. In an example, the first health-related event may be taking a medication and the first context may be morning. The computing device may identify that the user brushes his teeth every day without being monitored (e.g., without being notified or alerted). The computing device may group taking the morning mediation with teeth brushing such that a notification to take the morning medication is displayed to the user before, during, or after the user brushes their teeth. After a few times of being notified, taking the morning medication may become a high adherence ratio habit, partially due to being grouped with another high adherence ratio habit (e.g., teeth brushing). Additionally or alternatively, the computing device may group, at 908, the first and second health-related events within a calendar entry, a to-do list, and/or the like.

The computing device may detect, at 910, a triggering event associated with the first or second context. The triggering event may depend on the context. For example, the triggering event may be a time of day when the context is a time of day. The triggering event may be the user at a location when the context is a location. For example, the triggering event may be the user arriving home from work. The triggering event may be the completion of the first health-related event or some other health-related event.

Context may be used to adjust the notification timing for a specific health-related event. For example, under normal circumstances, a health-related event (e.g., normally performed at home) may be scheduled to be performed by the user in the evening, but geolocation data shows that the user is not at home (e.g., at a restaurant, at a store, at the movie theater, etc.). The computing device may adjust the notification timing and resulting acceptance criteria for the health-related event, for example, to allow the health-related event to be recorded later in the day (e.g., when the user is home).

The context may include micro-location information. The micro-location information may include specific rooms within a location (e.g., home, work, school, etc.). One or more devices such as pillboxes for medication adherence, Bluetooth beacons, and/or other smart home devices (e.g., lights, outlets, thermostats, security cameras, switches, door and occupancy sensors) may be used to provide micro-location information. For example, the computing device may request currently connected WiFi router names and GPS location that could be stored at the time of task completion, anonymized and categorized (e.g., internally or through APIs such as Foursquare, Google Place, etc.) as home, work/school, restaurant, and/or the like.

Historical timing data may be used to adjust the notification timing for a specific health-related event. For example, the health-related event may be associated with a predetermined time of day, but the user has logged completion of the health-related event at a different time of day. The computing device may adjust the notification timing and/or resulting acceptance criteria for the health-related event, for example, to allow the health-related event to be logged with the user logged completion in the past.

The computing device may communicate, at 912, a notification in response to detection of the triggering event. For example, the computing device may display, at 912, a notification associated with the notification group based on detection of the triggering event. The notification may be displayed to the user via an alert, a notification, a calendar entry, a to-do list, and/or the like.

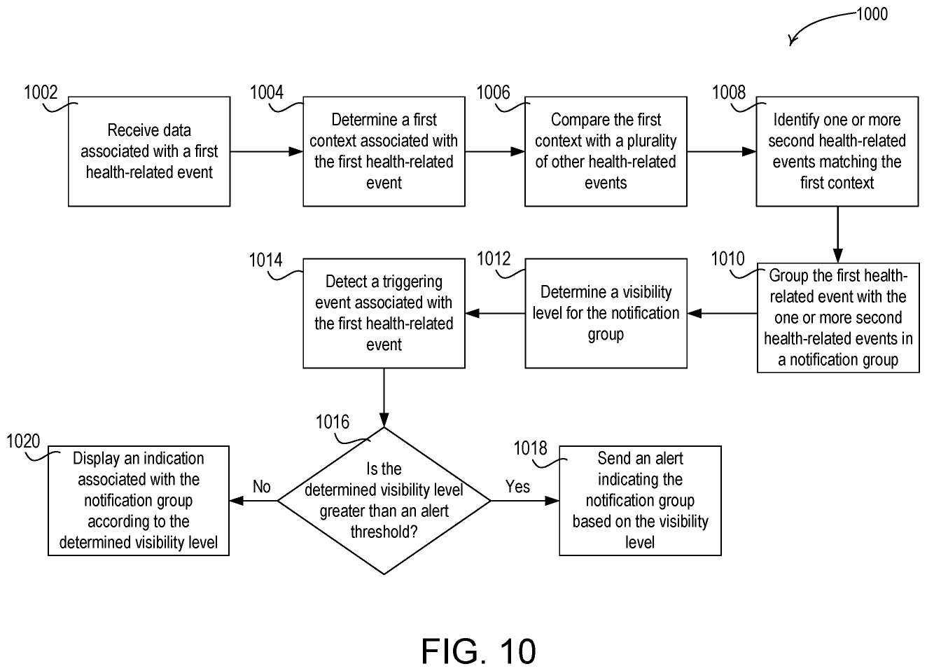

FIG. 10 shows a flowchart of another example process 1000 for determining whether to send an alert to a health-related event notification group. The example process 1000 is performed by one or more devices, such as one or more computing devices, for example. The process 1000 may be performed by a single computing device, or may be distributed across multiple devices. For example, the process 1000 may be performed by a computing device (e.g., such as mobile device 104 or remote computing device(s) 122 shown in FIG. 1). The example process 1000 may be performed by a control circuit (e.g., such as the processor 502 shown in FIG. 5) executing instructions from a memory (e.g., such as the memory 516 shown in FIG. 5). The process 1000 may be performed by a health management application (e.g., such as mobile application 105) executed by the computing device. Grouping health-related events into notification groups may reduce the number of notifications and/or interactions required by the user. For example, the computing device may display one notification for the notification group instead of one notification for each health-related event. In examples, the computing device may display time of day notifications (e.g., morning, afternoon, evening, bedtime, etc.) by notification group (e.g., morning notification group, afternoon notification group, evening notification group, bedtime notification group, etc.).

The computing device may receive, at 1002, data associated with a first health-related event. The data may include a manual entry indicating completion of the first health-related event. The manual entry may include a user response to a survey. In examples, the data may include an automated entry indicating completion of the first health-related event. For example, the computing device may determine that the first health-related event has been completed based on the data collected. Additionally or alternatively, the data collected, at 806, may include real-time data based on data received from other computing devices. For example, the computing device (e.g., the health management application) may link a blood glucose reading from a blood glucose monitor (e.g., the CGM 102 or the BGM 106 shown in FIG. 1) to the first health-related event. The computing device (e.g., the health management application) may link medication data for a medication and/or dietary data for a meal to the first health-related event based on a scan of a unique identifier associated with a medication and/or a meal. For example, at least a portion of the user-reported adherence data may be received by the computing device upon scanning one or more of a bar code, a quick response (QR) code, a radio frequency identification (RFID) tag, or a near field communication (NFC) tag attached to the medication or meal. The computing device may store the user-reported adherence data in the database.

The data may include one or more blood glucose levels, a dietary entry, a medication entry, an exercise entry, a body temperature, a heart rate, a blood pressure, an oxygen saturation level, sleep information, lab data, manual data entry, and/or the like. In examples, the data may include a manual entry indicating completion of the first health-related event. The manual entry may include a user response to a survey. In examples, the data may include an automated entry indicating completion of the first health-related event. For example, the computing device may determine that the first health-related event has been completed based on the data received, at 1002.

The computing device may determine, at 1004, a first context associated with the first health-related event. The first context may include a time, a location, one or more user characteristics and/or one or more event characteristics. The one or more user characteristics may include education level, socio-economic status, age, gender, marital status, and/or the like. The one or more event characteristics may include an activity code assigned to the first health-related event. In examples, each health-related event may be assigned a unique activity code. In other examples, a group of similar health-related events may be assigned a group activity code.

The computing device may compare, at 1006, the first context with a plurality of other health-related events that are associated with the user.

The computing device may identify, at 1008, one or more second health-related events that match the first context. In examples, the one or more second health-related events may have second contexts that are the same as the first context. In other examples, the second contexts may be similar to the first context.

The computing device may group, at 1010, the first health-related event and the one or more second health-related events into a notification group. For example, the first health-related event and the one or more second health-related events may be grouped into the notification group based on having similar contexts. Grouping health-related events into notification groups may reduce the number of notifications and/or interactions required by the user. For example, the computing device may display one notification for the notification group instead of one notification for each health-related event. Additionally or alternatively, grouping health-related events may increase adherence with one or more health-related events. For example, the second health-related event may be a habit having a high adherence ratio (e.g., greater than 80%). The high adherence ratio habit may be in a hidden notification visibility level and/or is performed by the user without being monitored or logged. Grouping the first health-related event with the high adherence ratio habit may make it easier for the user to remember to perform the first health-related event. In an example, the first health-related event may be taking a medication and the first context may be morning. The computing device may identify that the user brushes his teeth every day without being monitored. The computing device may group taking the morning mediation with teeth brushing such that a notification to take the morning medication is displayed to the user before, during, or after the user brushes their teeth. After a few times of being notified, taking the morning medication may become a high adherence ratio habit, partially due to being grouped with another high adherence ratio habit (e.g., teeth brushing). Additionally or alternatively, the computing device may group, at 908, the first and second health-related events within a calendar entry, a to-do list, and/or the like.

The computing device may determine, at 1012, a notification visibility level for the notification group. The computing device may determine, at 1012, a visibility level for the notification group based on the data received at 1002 and/or adherence data associated with the one or more second health-related events. Additionally or alternatively, the computing device may determine, at 1012, a visibility level for the notification group based on data associated with other users who have performed the first health-related event and/or the one or more second health-related events. The computing device may identify one or more (e.g., a group of) users having a similar age, gender, activity level, medical history, etc. The computing device may aggregate adherence data of the one or more users for the first health-related event and/or one or more second health-related events to determine an initial visibility level for the notification group. For example, the computing device may generate and/or maintain an adherence level model. The adherence level model may be used to determine the initial visibility level for the notification group for a user within an age range, having a specific gender, having a specific activity level, and/or specific medical history.

The computing device may detect, at 1014, a triggering event associated with the first health-related event. The triggering event may depend on the first context. For example, the triggering event may be a time of day when the first context is a time of day. The triggering event may be the user at a location when the first context is a location. For example, the triggering event may be the user arriving home from work. The triggering event may be the completion of another health-related event.

The computing device may determine, at 1016, whether the determined visibility level is greater than an alert threshold. For example, the computing device may compare the visibility level with the alert threshold. The alert threshold may be a predefined visibility level that triggers an alert to the user.

When the determined visibility level is greater than an alert threshold, the computing device may send, at 1018, an alert that indicates the notification group. For example, the computing device may communicate, at 1018, the alert in response to detection of the triggering event. For example, the alert may be audible or non-audible. A non-audible alert may be provided as a vibration, a flashing of the screen, and/or a flashing of an LED on the computing device. For example, the vibration may be accompanied by a notification displayed on the computing device. The notification may include an indication associated with the notification group. For example, the indication may be a reminder to perform and/or log completion of the health-related events of the notification group.

When the determined visibility level is less than the alert threshold, the computing device may display, at 1020, an indication associated with the notification group according to the determined visibility level. The indication may be a reminder to perform and/or log completion of the health-related events of the notification group.

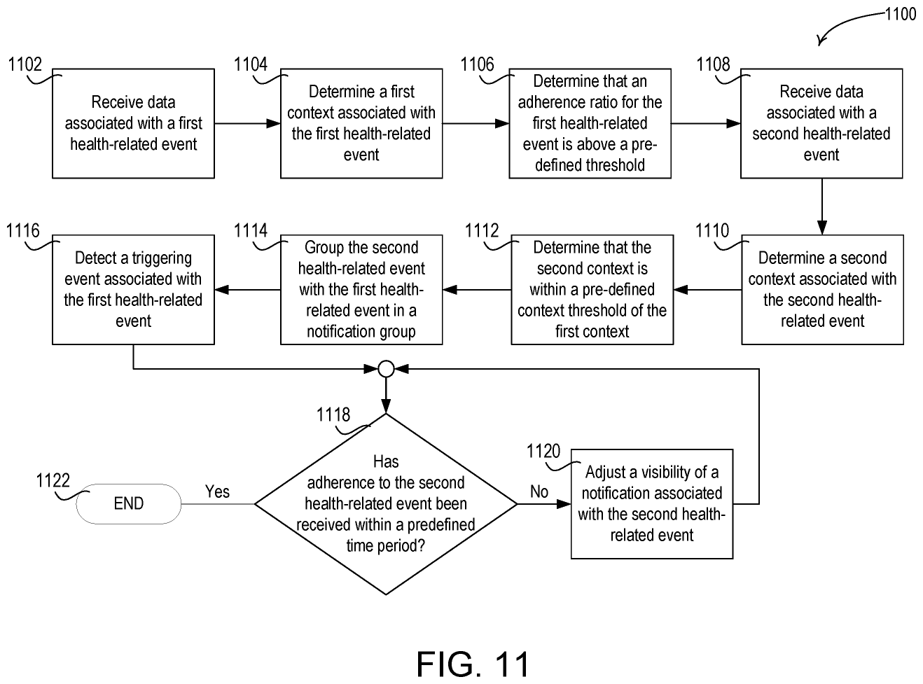

FIG. 11 shows a flowchart of another example process 1100 for adjusting a notification visibility level of a health-related event notification group. The example process 1100 is performed by one or more devices, such as one or more computing devices, for example. The process 1100 may be performed by a single computing device, or may be distributed across multiple devices. For example, the process 1100 may be performed by a computing device (e.g., such as mobile device 104 or remote computing device(s) 122 shown in FIG. 1). The process 1100 may be performed by a health management application (e.g., such as mobile application 105) executed by the computing device. The example process 1100 may be performed by a control circuit (e.g., such as the processor 502 shown in FIG. 5) executing instructions from a memory (e.g., such as the memory 516 shown in FIG. 5).

The computing device may receive, at 1102, data associated with a first health-related event. The data may include a manual entry indicating completion of the first health-related event. The manual entry may include a user response to a survey. In examples, the data may include an automated entry indicating completion of the first health-related event. For example, the computing device may determine that the first health-related event has been completed based on the data collected. Additionally or alternatively, the data received, at 1102, may include real-time data based on data received from other computing devices. For example, the computing device (e.g., the health management application) may link a blood glucose reading from a blood glucose monitor (e.g., the CGM 102 or the BGM 106 shown in FIG. 1) to the first health-related event. The computing device (e.g., the health management application) may link medication data for a medication and/or dietary data for a meal to the first health-related event based on a scan of a unique identifier associated with a medication and/or a meal. For example, at least a portion of the data may be received by the computing device upon scanning one or more of a bar code, a quick response (QR) code, a radio frequency identification (RFID) tag, or a near field communication (NFC) tag attached to the medication or meal. The computing device may log the data, for example, in the database.

The received data may include one or more blood glucose levels, a dietary entry, a medication entry, an exercise entry, a body temperature, a heart rate, a blood pressure, an oxygen saturation level, sleep information, lab data, manual data entry, and/or the like. In examples, the received data may include a manual entry indicating completion of the first health-related event. The manual entry may include a user response to a survey. In examples, the received data may include an automated entry indicating completion of the first health-related event. For example, the computing device may determine that the first health-related event has been completed based on the data received, at 1102.

The computing device may determine, at 1104, a first context associated with the first health-related event. The first context may include a time, a location, one or more user characteristics and/or one or more event characteristics. The one or more user characteristics may include education level, socio-economic status, age, gender, marital status, and/or the like. The one or more event characteristics may include an activity code assigned to the first health-related event. In examples, each health-related event may be assigned a unique activity code. In other examples, a group of similar health-related events may be assigned a group activity code.

The computing device may determine, at 1106, that an adherence ratio for the first health-related event is above a pre-defined threshold. The pre-defined threshold may be associated with a high adherence level (e.g., such as adherence level 4 shown in Table 1. For example, the pre-defined threshold may be a lower adherence threshold for the high adherence level. The adherence ratio may be determined based on the received data associated with the health-related event. The adherence ratio may be a quantitative measure of how frequently the user performs the health-related event in a given period of time (e.g., an adherence period). Stated differently, the adherence ratio may indicate the user's historical compliance with completion of the health-related event over the adherence period. The adherence ratio may be determined using the number of opportunities to perform the health-related event during the adherence period and the number of completions of the health-related event during the adherence period.

The computing device may receive, at 1108, data associated with a second health-related event. The data may include a manual entry indicating completion of the second health-related event. The manual entry may include a user response to a survey. In examples, the data may include an automated entry indicating completion of the second health-related event. For example, the computing device may determine that the second health-related event has been completed based on the received data. Additionally or alternatively, the data received, at 1108, may include real-time data based on data received from other computing devices. For example, the computing device (e.g., the health management application) may link a blood glucose reading from a blood glucose monitor (e.g., the CGM 102 or the BGM 106 shown in FIG. 1) to the second health-related event. The computing device (e.g., the health management application) may link medication data for a medication and/or dietary data for a meal to the second health-related event based on a scan of a unique identifier associated with a medication and/or a meal. For example, at least a portion of the user-reported adherence data may be received by the computing device upon scanning one or more of a bar code, a quick response (QR) code, a radio frequency identification (RFID) tag, or a near field communication (NFC) tag attached to the medication or meal. The computing device may store the user-reported adherence data in the database.

The received data may include one or more blood glucose levels, a dietary entry, a medication entry, an exercise entry, a body temperature, a heart rate, a blood pressure, an oxygen saturation level, sleep information, lab data, manual data entry, and/or the like. In examples, the received data may include a manual entry indicating completion of the second health-related event. The manual entry may include a user response to a survey. In examples, the received data may include an automated entry indicating completion of the second health-related event. For example, the computing device may determine that the second health-related event has been completed based on the data received, at 1108.

The computing device may determine, at 1110, a second context associated with the second health-related event. The second context may include a time, a location, one or more user characteristics and/or one or more event characteristics. The one or more user characteristics may include education level, socio-economic status, age, gender, marital status, and/or the like. The one or more event characteristics may include an activity code assigned to the second health-related event. In examples, each health-related event may be assigned a unique activity code. In other examples, a group of similar health-related events may be assigned a group activity code.

The computing device may determine, at 1112, that the second context is within a pre-defined context threshold of the first context. The pre-defined context threshold may define a relative difference threshold between similar contexts. The pre-defined context threshold may be based on the context type. For example, when the context type is time, the pre-defined context threshold may be a length of time (e.g., such as 5 minutes, 15 minutes, etc.); when the context type is location, the pre-defined context threshold may be a distance (e.g., 100 yards, 1 mile, etc.); etc.

The computing device may group, at 1114, the first health-related event and the second health-related event into a notification group. For example, the first health-related event and the second health-related event may be grouped into the notification group based on second context being within the pre-defined threshold of the first context. Grouping health-related events into notification groups may reduce the number of notifications and/or interactions required by the user. For example, the computing device may display one notification for the notification group instead of one notification for the first health-related event and another notification for the second health-related event. Additionally or alternatively, grouping health-related events may increase adherence with the second health-related event. For example, the second health-related event may be grouped with the first health-related event because the first health-related event has a high adherence ratio (e.g., greater than 80%). The high adherence ratio habit may be in a hidden notification visibility level and/or is performed by the user without being monitored or logged. Grouping the second health-related event with the first health-related event may make it easier for the user to remember to perform the second health-related event.

The computing device may detect, at 1116, a triggering event associated with the first health-related event. The triggering event may depend on the first context. For example, the triggering event may be a time of day when the first context is a time of day. The triggering event may be the user at a location when the first context is a location. For example, the triggering event may be the user arriving home from work. The triggering event may be the completion of another health-related event. The computing device may communicate a notification in response to detection of the triggering event.

The computing device may determine, at 1118, whether adherence to the second health-related event has been received within a predefined time period. The predefined time period may be a standard time delay for reporting completion of the second health-related event. The time delay may start when completion of the second health-related event is expected. The predefined time period may be separately determined for each health-related event or generally determined for the health-related events. Additionally or alternatively, the predefined time period may be determined based on context. For example, each context may be associated with a specific predefined time period for adherence reporting.

When adherence to the second health-related event has not been received within the predefined time period, the computing device may adjust, at 1120, a visibility of a notification associated with the second health-related event (e.g., the notification group). For example, the visibility of the notification associated with the second health-related event may be increased such that the likelihood that the user sees the notification is increased. Stated differently, the second health-related event may be associated with an initial notification visibility level, as described herein. The initial notification visibility level of the second health-related event may be adjusted to the next highest notification visibility level when adherence is not received within the predefined time period. For example, expiration of the predefined time period (e.g., time delay) may trigger switching from display of a notification to an audible or non-audible alert. A non-audible alert may be provided as a vibration, a flashing of the screen, and/or a flashing of an LED on the computing device. For example, the vibration may be accompanied by a notification displayed on the computing device. The notification may include an indication associated with the notification group. For example, the indication may be a reminder to perform and/or log completion of the health-related events of the notification group.

After adjustment of the notification visibility level, the computing device may send and/or display a notification according to the adjusted notification visibility level to the user. The predefined time period may start again following sending and/or displaying of the notification according to the adjusted notification visibility level. The process 1100 may proceed back to 1118 after sending and/or displaying the notification at the adjusted notification visibility level. The computing device may again determine, at 1118, whether adherence to the second health-related event is received after sending and/or displaying the notification at the adjusted notification visibility level. The visibility of the notification may continue to be adjusted until the user reports adherence to the second health-related event and/or a maximum visibility level is achieved.

When adherence to the second health-related event has been received within the predefined time period, the process 1100 may end at 1122.

Although features, elements, and functions are described above in particular combinations, a feature, element, or function is used alone or in any combination with the other features, elements, or functions. Various presently unforeseen or unanticipated alternatives, modifications, variations, or improvements may be subsequently made that are also intended to be encompassed by the following claims.

The methods described herein are implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), removable disks, and optical media such as CD-ROM disks, and digital versatile disks (DVDs).

What is claimed is:

1. A method comprising:
identifying a user health-related event associated with a user, wherein the user health-related event is a diabetes related event;
determining a context associated with the user health-related event, wherein the context comprises at least one of a time, a location, or a medical condition associated with the user health-related event;
determining a first notification visibility level for the user health-related event, wherein the first notification visibility level is a measure of the visibility to the user of a notification associated with the user health-related event;
receiving data associated with the user health-related event;
determining an adherence level for the user health-related event based on the data associated with the user health-related event;
comparing the adherence level for the user health-related event to a plurality of predefined adherence thresholds;
in response to comparing the adherence level to the plurality of predefined adherence thresholds, determining a second notification visibility level for the user health-related event;
detecting a triggering event associated with the user health-related event;
communicating, using the second notification visibility level, the notification associated with the user health-related event to a mobile device associated with the user based on detection of the triggering event.

2. The method of claim 1, wherein the first notification visibility level is determined based on the context.

3. The method of claim 1, wherein the second notification visibility level is determined to be a higher visibility level than the first notification visibility level when the adherence level is below a first predefined adherence threshold associated with the first notification visibility level.

4. The method of claim 1, wherein the second notification visibility level is determined to be a lower visibility level than the first notification visibility level when the adherence level is greater than a second predefined adherence threshold associated with the first notification visibility level.

5. The method of claim 1, wherein the first and second notification visibility levels comprise an intervention visibility level, a request input visibility level, a dashboard visibility level, or a hidden visibility level.

6. The method of claim 5, wherein the intervention visibility level comprises communicating the notification to the mobile device of the user and to at least one other device.

7. The method of claim 5, wherein the request input visibility level comprises requesting the user to log completion of the user health-related event.

8. The method of claim 5, wherein the dashboard visibility level comprises displaying a list of the user health-related event and at least one other user health-related event to be completed in a given time period.

9. The method of claim 8, wherein the list is organized based on context.

10. The method of claim 5, wherein the hidden visibility level comprises hiding the notification from the user.

11. The method of claim 1, wherein the request input visibility level is selected as the first notification visibility level when the user health-related event is new to the user.

12. The method of claim 1, further comprising:
determining that the context associated with the user health-related event matches the context of at least one other user health-related event, wherein the at least one other user health-related event is a non-diabetes related event; and
grouping the user health-related event with the at least one other user health-related event in a notification group, wherein the notification group is used to send a notification that indicates the user health-related event and the at least one other user health-related event.

13. The method of claim 1, wherein the adherence level comprises a range of adherence for the user health-related event.

14. The method of claim 1, wherein the user health-related event is associated with a plurality of adherence levels.

15. The method of claim 14, wherein each of the plurality of adherence levels comprises an upper adherence threshold and a lower adherence threshold.

16. The method of claim 14, wherein the plurality of adherence levels are adjustable based on one or more of a characteristic of the user, an age of the user health-related event, a characteristic of the user health-related event, a number of times adherence of the user health-related event has been logged within a predefined time period, or the frequency of the user health-related event.

17. The method of claim 1, wherein at least a portion of the data is received by the mobile device of the user upon scanning one or more of a bar code, a quick response (QR) code, a radio-frequency identification (RFID) tag, or a near field communication (NFC) tag.

18. A method comprising:
logging a plurality of entries associated with a first user health-related event in a memory of a mobile device associated with a user, wherein the first user health-related event comprises one of a diabetes-related event or a non-diabetes related event;
determining a first context associated with the first user health-related event, wherein the first context comprises at least one of a time or a location associated with the first user health-related event;
determining that the plurality of entries indicate that the user is above an adherence threshold for the first user health-related event;
logging a second user health-related event, wherein the second user health-related event comprises a diabetes-related event;
determining a second context associated with the second user health-related event, wherein the second context comprises at least one of a time or a location associated with the second user health-related event;
determining that the first context associated with the first user health-related event is within a predefined threshold of the second context associated with the second user health-related event;
in response to determining that the context associated with the first user health-related event is within the predefined threshold, grouping the first user health-related event with the second user health-related event in a notification group;
detecting a triggering event when user is estimated to perform the first user-health related event; and
communicating, via an audible alert or a visible alert on the mobile device, an alert to the user to perform at least the second user health-related event based on detection of the triggering event.

19. The method of claim 18, further comprising adjusting a visibility of the alert in response to a number of times the second user health-related event is logged within a predefined period of time and the adherence threshold.

20. The method of claim 18, wherein the alert is communicated based on the triggering event being detected for the first user-health related event, and wherein the first user-health related event being above the adherence threshold causes the visible alert or audible alert to fail to include information related specifically to the first user-health related event.

21. The method of claim 18, wherein the alert is communicated at a notification visibility level which is determined based on the context.

22. The method of claim 21, wherein the notification visibility level comprises an intervention visibility level, a request input visibility level, a dashboard visibility level, or a hidden visibility level.

23. The method of claim 22, wherein the intervention visibility level comprises communicating the notification to the mobile device of the user and to at least one other device.

24. The method of claim 22, wherein the request input visibility level comprises requesting the user to log completion of the user health-related event.

25. The method of claim 22, wherein the dashboard visibility level comprises displaying a list of the user health-related event and at least one other user health-related event to be completed in a given time period.

26. The method of claim 25, wherein the list is organized based on context.

27. The method of claim 22, wherein the hidden visibility level comprises hiding the notification from the user.

28. The method of claim 18, wherein the request input visibility level is selected as the first notification visibility level when the user health-related event is new to the user.

29. The method of claim 18, wherein the user health-related event is associated with a plurality of adherence levels.

30. The method of claim 29, wherein each of the plurality of adherence levels comprises an upper adherence threshold and a lower adherence threshold.

31. The method of claim 29, wherein the plurality of adherence levels are adjustable based on one or more of a characteristic of the user, an age of the user health-related event, a characteristic of the user health-related event, a number of times adherence of the user health-related event has been logged within a predefined time period, or the frequency of the user health-related event.

32. The method of claim 18, wherein the data is received by the mobile device of the user upon scanning one or more of a bar code, a quick response (QR) code, a radio-frequency identification (RFID) tag, or an near field communication (NFC) tag.

33. At least one computer-readable storage medium having computer-executable instructions stored thereon that are configured to, when executed by at least one processor, cause the at least one processor to:
identify a user health-related event associated with a user, wherein the user health-related event is a diabetes related event;
determine a context associated with the user health-related event, wherein the context comprises at least one of a time, a location, or a medical condition associated with the user health-related event;
determine a first notification visibility level for the user health-related event, wherein the first notification visibility level is a measure of the visibility to the user of a notification associated with the user health-related event;
receive data associated with the user health-related event;
determine an adherence level for the user health-related event based on the data associated with the user health-related event;
compare the adherence level for the user health-related event to a plurality of predefined adherence thresholds;
in response to the comparison of the adherence level to the plurality of predefined adherence thresholds, determine a second notification visibility level for the user health-related event;
detect a triggering event associated with the user health-related event;
communicate, using the second notification visibility level, the notification associated with the user health-related event to a mobile device associated with the user based on detection of the triggering event.

34. The at least one computer-readable storage medium of claim 33, wherein the instructions are configured to cause the processor to determine the first notification visibility level based on the context.

35. The at least one computer-readable storage medium of claim 33, wherein the instructions are configured to cause at least one processor to determine the second notification visibility level to be a higher visibility level than the first notification visibility level when the adherence level is below a first predefined adherence threshold associated with the first notification visibility level.

36. The at least one computer-readable storage medium of claim 33, wherein the instructions are configured to cause at least one processor to determine the second notification visibility level to be a lower visibility level than the first notification visibility level when the adherence level is greater than a second predefined adherence threshold associated with the first notification visibility level.

37. The at least one computer-readable storage medium of claim 33, wherein the first and second notification visibility levels comprise an intervention visibility level, a request input visibility level, a dashboard visibility level, or a hidden visibility level.

38. The at least one computer-readable storage medium of claim 37, wherein the intervention visibility level is configured to communicate the notification to the mobile device of the user and to at least one other device.

39. The at least one computer-readable storage medium of claim 37, wherein the request input visibility level is configured to request the user to log completion of the user health-related event.

40. The at least one computer-readable storage medium of claim 37, wherein the dashboard visibility level is configured to display a list of the user health-related event and at least one other user health-related event to be completed in a given time period.

41. The at least one computer-readable storage medium of claim 40, wherein the list is organized based on context.

42. The at least one computer-readable storage medium of claim 37, wherein the hidden visibility level is configured to hide the notification from the user.

43. The at least one computer-readable storage medium of claim 33, wherein the instructions are configured to cause at least one processor to select the request input visibility level as the first notification visibility level when the user health-related event is new to the user.

44. The at least one computer-readable storage medium of claim 33, wherein the instructions are further configured to cause the processor to:
determine that the context associated with the user health-related event matches the context of at least one other user health-related event, wherein the at least one other user health-related event is a non-diabetes related event; and
group the user health-related event with the at least one other user health-related event in a notification group, wherein the notification group is configured to be used to send a notification that indicates the user health-related event and the at least one other user health-related event.

45. The at least one computer-readable storage medium of claim 33, wherein the adherence level comprises a range of adherence for the user health-related event.

46. The at least one computer-readable storage medium of claim 33, wherein the user health-related event is associated with a plurality of adherence levels.

47. The at least one computer-readable storage medium of claim 46, wherein each of the plurality of adherence levels comprises an upper adherence threshold and a lower adherence threshold.

48. The at least one computer-readable storage medium of claim 46, wherein the plurality of adherence levels are adjustable based on one or more of a characteristic of the user, an age of the user health-related event, a characteristic of the user health-related event, a number of times adherence of the user health-related event has been logged within a predefined time period, or the frequency of the user health-related event.

49. The at least one computer-readable storage medium of claim 33, wherein at least a portion of the data is received by the mobile device of the user upon scanning one or more of a bar code, a quick response (QR) code, a radio-frequency identification (RFID) tag, or a near field communication (NFC) tag.
